(19) United States
(12) Patent Application Publication
(10) Pub. No.: US 2011/0152371 A1
(43) Pub. Date: Jun. 23, 2011

(54) ANTIOXIDANT EXTRACT FROM FRUIT SKINS

(76) Inventors: Vasantha Handukutti Pathirannehalage Rupasinghe, Truro (CA); Marie Gwendolyn Huber, London (CA); Afsana Yasmin, Truro (CA); Naciye Erkan, Truro (CA)

(21) Appl. No.: 12/808,574

(22) PCT Filed: Dec. 19, 2008
(86) PCT No.: PCT/CA2008/002261

§ 371 Date: Feb. 28, 2011

Related U.S. Application Data
(60) Provisional application No. 61/014,958, filed on Dec. 19, 2007.

(51) Int. Cl. A61K 31/20; A61K 31/05; A61P 9/00

(52) U.S. Cl. 514/560; 514/731

(57) ABSTRACT

The application generally relates to methods of inhibition of oxidation of polyunsaturated fatty acid—(PUFA) and/or lipid-containing foods and nutraceutical products. The application provides natural and consumer friendly method for preventing the oxidation or rancidity development of PUFA and/or lipids by incorporation of apple skin extracts as natural antioxidant in emulsions, bulk oil or other form of food and nutraceutical products.

### FIELD OF THE APPLICATION

The present application generally relates to a method for inhibiting the oxidation of polyunsaturated fatty acids or lipids using a novel, naturally occurring mixture of antioxidants extracted from fruit, in particular apple skins, a by-product of food processing.

### BACKGROUND OF THE APPLICATION

Dietary lipids and fatty acid profiles and their balance within the body have become one of the interesting areas of recent investigations since lower levels of endogenous omega-3 fatty acids have been implicated to several chronic diseases (Simopoulos, 2002). Within omega-3 fatty acids, α-linolenic acid (LNA, C18:3 n-3), eicosapentaenoic acid (EPA, C20:5 n-3) and docosahexaenoic acid (DHA, C22:6 n-3) are the most important long chain polyunsaturated fatty acids (PUFA) with strong scientific evidence for their potential to reduce the risk of cardiovascular disease (Wang et al., 2006), inflammatory effects such as rheumatoid arthritis (Kremer, 2000) and various cancers (Wigmore et al., 1996). In addition, fish oil is the vital source of EPA and DHA in human diet.

As a result, omega-3 fatty acid containing functional foods and nutraceuticals have been introduced to the market. However, the presence of multiple double bonds of PUFA makes them vulnerable to oxidation, which produces various aldehydes and ketones resulting in unacceptable colours, odours, and flavours in PUFA containing foods and nutraceutical products (Nawar, 1996). Moreover, the products of lipid oxidation, such as malonaldehyde, can have adverse health effects to the consumer due to their cytotoxic and genotoxic effects (Esterbauer et al., 1990; Fang et al., 1996). The high rate of oxidation of PUFA can be controlled by the addition of synthetically produced antioxidants such as butylated hydroxytoluene (BHT), butylated hydroxyanisole (BHA), and synthetic or naturally sourced α-tocopherol. Although the synthetic antioxidants possess powerful protective ability against oil decomposition, potential carcinogenic properties of the synthetic antioxidants have been reported (Botterweck et al., 2000; Amarowicz, 2000) and use of them in food has been already limited in some countries. Recently, consumer health consciousness has led to a demand for ‘natural’ alternatives to synthetically produced food antioxidants such as the butylated hydroxyl compounds, BHT and BHA.

Natural plant-based antioxidants such as phenolics derived from fruits, vegetables and many herbal or aromatic plants have received much attention for their antioxidative characteristics. Many phytochemicals are also important dietary antioxidants and cell signaling modulators in preventing oxidative stress mediated degenerative diseases (Kaur and Kapoor, 2001). Apple is a great source of natural antioxidant in the North American and European diet and provides about 22% of total dietary phenolics (Vinson et al 2001). Apple skin has 3 to 6-fold higher flavonoid content than apple flesh and has unique flavonoids, such as quercetin glycosides, not found in the flesh (Wolfe, Wu, & Liu, 2003; Wolfe & Liu, 2003). The apple skin extract has been shown to possess powerful free radical scavenging activity (Kondo et al.; 2002). It has been estimated that 2-3 million kg of apple skins are generated as a result of apple processing in Nova Scotia, Canada (Rupasinghe 2003).

Phytochemical rich extract from other food and food ingredient sources such as oregano (Tsimidou et al., 1995), barley bran (Katsanidis et al., 1997), green tea (Wanasundara and Shahidi 1998), borage seed (Wettasinghe and Shahidi 1999), rosemary (Montero et al., 2005; Erkan et al., 2008), grape seed extract (Pazos et al, 2005), garlic (Iqbal and Bhanger 2007), leaves of Mallotus Japonicus (Tabata et al, 2008), and leaves of Smilax excelsa (Ozsoy et al., 2008) have show to possess the ability of inhibition of lipid oxidation in various model systems. Tsimidou and coworker (1995) used dry oregano (1% w/v) to prevent mackerel oil oxidation at 40° C. storage condition that yields activity equivalent to 200 ppm synthetic antioxidant, TBHQ. Wanasundara and Shahidi (1998) evaluated the potency of green tea extracts for the protection of onset oxidation in a number of marine oils by which presented as superior than those of natural (α-tocopherol) and synthetic antioxidants (BHA and BHT). Significant protection has been found by rosemary extract on the oxidative stabilization of corn oil (Frankel, 1998). However, though many of the plant extracts exhibit ability for inhibition of lipid peroxidation, the characteristic smell and flavor due to their incorporation has raised concerns for their use as alternatives of synthetic antioxidants.

### SUMMARY OF THE APPLICATION

In the present study, apple skin extracts were prepared and their anti-oxidant efficacy compared with the commonly used natural and synthetic antioxidants, α-tocopherol, butylated hydroxytoluene (BHT) and tert-butyl hydroquinone (TBHQ). The ability of the apple skin extracts to inhibit oxidation of methyl linolinate (ML), eicosapentaenoic acid (EPA), and docosahexaenoic acid (DHA) were studied using oil-in-water emulsion system under three different induction systems, heat, peroxyl radical and UV light. Evaluations were also extended to omega-3 enriched fish oil in bulk system. For the first time, the omega-3 fatty acid or lipid preserving potential of apple skin extracts is reported herein. Accordingly, the phenolics isolated from apple skin represent a natural alternative to synthetic antioxidants for the stabilization of omega-3 fatty acid containing food and nutraceuticals.

Accordingly, the present application relates to a method of preventing or inhibiting the oxidation of polyunsaturated fatty acids and/or lipids comprising contacting the polyunsaturated fatty acids and/or lipids with an effective amount of an extract comprising phenolic compounds from fruit skins, in particular apple skins.

In an embodiment of the application, the phenolic compounds were obtained from the fruit skins by extracting a sample of the skins with a food-grade organic solvent, in particular, ethanol. The resulting solution was centrifuged and the resulting supernatant was the antioxidant extract. Accordingly, in another embodiment of the present application, there is included a process for extracting plant phenolic compounds from fruit, in particular apples, comprising:

(a) obtaining a sample of fruit peels,

(b) optionally dehydrating the peels and converting the peels into a powder;

(c) extracting the peels with a food-grade solvent under conditions to extract the plant phenolic compounds into the solvent; and

(d) removing solids from the extract of (c) to provide a stock solution of extracted plant phenolic compounds.

In further embodiments the stock solution of extracted plant phenolic compounds obtained from the above-described process is reduced to dryness to provide a solid concentrate of extracted plant phenolic compounds. In a still further embodiment, the resulting solid concentrate is taken up into water to provide an aqueous solution having insoluble suspended material and this insoluble material is removed, for example by centrifuging or filtering, to provide a clear aqueous solution of extracted plant phenolic compounds.

In an embodiment of the application the stock solution of extracted plant phenolic compounds, the solid concentrate of extracted plant phenolic compounds or the aqueous solution of extracted plant phenolic compounds is treated to remove sugar compounds. In another embodiment the sugars are removed by chromatography. The extracted plant phenolic compounds are typically removed from the column by flushing the column with a food-grade solvent and the resulting phenolic compound-containing solution is used as is or the solvent is removed to provide a solid concentrate of extracted plant phenolic compounds that is essentially sugar-free. This solid concentrate is, again, used as is, freeze dried for storage or taken up in another solvent for use as a stock solution of extracted plant phenolic compounds.

In a further embodiment of the present application a food-grade carrier is added to the stock solution of extracted plant phenolic compounds, the solid concentrate of extracted plant phenolic compounds or the aqueous solution of extracted plant phenolic compounds or the essentially sugar-free versions thereof.

In another embodiment of the application, the resulting extracts of plant phenolic compounds, in the form of a solution in a food-grade organic solvent or in water, or in the form of a solid, with or without a carrier, is added to any solid or liquid sample comprising PUFA's and/or lipids, including, for example, any food, feed, nutraceutical product and cosmetic product.

Other features and advantages of the present application will become apparent from the following detailed description. It should be understood, however, that the detailed description and the specific examples while indicating preferred embodiments of the application are given by way of illustration only, since various changes and modifications within the spirit and scope of the application will become apparent to those skilled in the art from this detailed description.

### BRIEF DESCRIPTION OF THE DRAWINGS

The disclosure will now be described in relation to the drawings in which:

FIG. 1 illustrates the distribution of major phenolic groups among three collections of apples including commercial cultivars, new breeding lines, and crab apples/heritage cultivar, in an embodiment of the disclosure; and

FIG. 2 illustrates the concentration of phenolic compounds (mg/100 g DW) in apple skin for 2005 and 2006 collection of apples, in an embodiment of the disclosure.

### DETAILED DESCRIPTION OF THE APPLICATION

### Definitions

The following terms are used throughout the application and their meaning provided below is meant to apply to each embodiment of the present application.

The terms “phenolics”, “fruit phenolics” or “plant-phenolics” are used herein substantially interchangeably and in the manner normally used in food chemistry and related art. That is, these terms refer to non-toxic substances naturally occurring in plants (primarily in fruits, in particular apples) and which have an aromatic hydroxyl group and react like gallic acid in various reactions and assays, such as the art-accepted Folin-Ciocalteau reaction or assay. Gallic acid is 3,4,5-trihydroxybenzoic acid, and the Folin-Ciocalteau reaction or assay is commonly used in the art to quantitatively measure phenolics, the amount or concentration of which is expressed in terms of equivalents to gallic acid (Gallic Acid Equivalent per liter; GAE/I). The phenolic compounds included in fruits, and extracted there from in accordance with the present application include, for example, phenolic acids, flavan-3-ols, flavonols, phloridzin, cinnamates, hydroxymethyl furfural, dihydroxychalcones, proanthocyanidins and anthocyanins.

The term “effective amount of an extract comprising phenolic compounds” as used herein is an amount to provide about 1 ppm to about 20,000 ppm, suitably between about 2 ppm and about 10,000 pm, more suitably between about 2 ppm and about 5000 ppm (ppm=mg/L) of total phenolics in a product comprising the polyunsaturated fatty acids (PUFA) and/or lipids, for example an aqueous emulsion or bulk oil or any other form of polyunsaturated fatty acids (PUFA) or PUFA containing lipids.

The term “fatty acid” as used herein refers to carboxylic acids with a long chain containing at least 8 carbon atoms. PUFAs contain two or more cis double bonds in the carbon chain. The PUFA or lipid may be any such compound found in a source in which it is desirable to inhibit its oxidation. For example, the PUFA or lipid is comprised in any nutraceutical (natural health product) or cosmetic product containing polyunsaturated fatty acids (including omega-3, 6 and 9) and/or their corresponding lipids. The product may be in the form of an emulsion, oil, cream, solid, liquid, or it may be a microencapsulated product. The food may be either for human or animal consumption.

The term lipids or “fats” as used herein generally refers to esters of glycerol and fatty acids

The term “dehydration” or “dehdrate” as used herein refers to any method of removing liquid from a sample, including for example, freeze-drying, air-drying, vacuum-drying, oven-drying, or any other form of drying.

In understanding the scope of the present disclosure, the term “comprising” and its derivatives, as used herein, are intended to be open ended terms that specify the presence of the stated features, elements, components, groups, integers, and/or steps, but do not exclude the presence of other unstated features, elements, components, groups, integers and/or steps. The foregoing also applies to words having similar meanings such as the terms, “including”, “having” and their derivatives. Finally, terms of degree such as “substantially”, “about” and “approximately” as used herein mean a reasonable amount of deviation of the modified term such that the end result is not significantly changed. These terms of degree should be construed as including a deviation of at least ±5% of the modified term if this deviation would not negate the meaning of the word it modifies.

### METHODS OF THE APPLICATION

Apples are a rich source of phenolic compounds, particularly apple skins (or peels), and contain a mixture of many flavonoids. In Nova Scotia, apple skins are available year-round (2-3 million kg per year) as a co-product of the apple processing industry (Rupasinghe, 2003). Therefore, phenolics isolated from apple skin represent an ideal source of natural antioxidants for the food industry. In accordance with the present application, naturally occurring phenolics are extracted from fruit peels, in particular apple peels, and a liquid or solid product is obtained which is significantly enriched in phenolics and which is utilized as an additive to various and diverse food items to provide the food item with a significant quantity of phenolics originating from the fruit. In particular the liquid or solid product enriched in plant phenolic compounds is used to inhibit or prevent the oxidation of polyunsaturated fatty acids (PUFA) and/or lipids.

The present application, therefore, relates to a method of preventing or inhibiting the oxidation of PUFA and/or lipids comprising contacting the PUFA and/or lipids with an effective amount of an extract comprising phenolic compounds from fruit skins, in particular apple skins.

It is an embodiment that the phenolic compounds are obtained from apple peels or skins. The apple may be any genotype of apple (Malus domestica) or crab apple (wild types).

In another embodiment of the application the PUFA and/or lipids are comprised in any nutraceutical (natural health product) or cosmetic product containing PUFA and/or lipids. In a further embodiment, the food, feed, nutraceutical or cosmetic product is in the form of emulsions, oils, liquids, solids, creams, or a microencapsulated product. In a further embodiment the polyunsaturated fatty acids and/or lipids are comprised in a food for animal or human consumption. In another embodiment, the product is an oil-in-water emulsion, such as soups, salad dressings, and sauces, or a bulk oil, such as fish oil.

In a further embodiment, the extract comprising phenolic compounds has been treated under conditions to remove sugar compounds. In yet another embodiment, the extract comprising phenolic compounds has been treated under conditions to remove lipids, carontenoids, chlorophylls and/or proanthocyanidins.

In another embodiment of the present application, there is included a process for extracting plant phenolic compounds from fruit, in particular apples, comprising:

(a) obtaining a sample of fruit peels,

(b) optionally dehydrating the peels and converting the peels into a powder;

(c) extracting the peels with a food-grade solvent under conditions to extract the plant phenolic compounds into the solvent; and

(d) removing solids from the extract from (c) to provide a stock solution of plant phenolic compounds.

In an embodiment the peels are either dehydrated or soaked in a salt solution, for example calcium chloride, as soon as possible after peeling from the fruit, for example within 10 minutes of peeling, thereby preserving the antioxidant compounds present in the peels. The peels soaked in salt solution are either extracted directly or freeze-dried for storage and/or transport.

In an embodiment of the application, dehydrated peels are converted into a fine powder using mechanical grinding means, such as a coffee grinder or an industrial equivalent.

In a further embodiment of the application, the food-grade solvent is ethanol, for example about 40% to about 100% ethanol, suitably 100% ethanol. In another embodiment of the application the conditions to extract the plant phenolic compounds into the solvent comprise sonicating for a sufficient period of time, for example about 5 minutes to 2 hours, suitably about 10 minutes to about 30 minutes. In a further embodiment of the application, the solids are removed from the extract by centrifuging.

In another embodiment of the disclosure, the peels are soaked in a salt solution, for example calcium chloride, at a temperature of about 50° C. to about 70° C., in particular at about 60° C., for about 5 to about 30 minutes, in particular 10 minutes. In a further embodiment, skins are frozen for later use or are ground into a slurry, using for example an Ursher Mill. In another embodiment, the apple peel slurry is then extracted using a food-grade solvent, for example ethanol at a concentration of about 40% to about 100%, in particular 100%. The extraction process is aided using an ultrasonication bath, for example at 20 kHz for about 5 minutes to about 60 minutes, in particular for 30 about minutes. The resulting solids are then separated using centrifugation or any other method.

In another embodiment of the disclosure, salt soaked apple peels are dried in an oven with air circulation at a temperature of about 50° C. to about 70° C., in particular at about 60° C., for about 24 to 72 hours, in particular for about 48 hours. In another embodiment, the dried peels are ground into a fine powder using a mechanical grinding means, such as a coffee grinder or an industrial equivalent. In a further embodiment, the ground peels are extracted with a food-grade solvent, for example, ethanol at a concentration of about 40% to about 100%, in particular 95%. In another embodiment the conditions to extract the plant phenolic compounds into the solvent comprise sonicating for a sufficient period of time, for example about 5 minutes to 2 hours, suitably about 10 minutes to about 30 minutes. In a further embodiment of the application, the solids are removed from the solution of extracted phenolic compounds by centrifuging.

In another embodiment of the disclosure, the method further comprises removing proanthocyanidins, lipids, carontenoids and/or chlorophylls from the peels by extracting the peels with hexane (to remove lipids, carontenoids and/or chlorophylls) and/or by extracting the peels with a mixture of acetone, water and acetic acid (to remove proanthocyanidins). In an embodiment, the extraction is performed by adding the peels or peel extract to the solvent and sonicating to facilitate dissolution of the desired materials and the remaining solids collected by filtration and/or centrifuging.

In further embodiments the stock solution of plant phenolic compounds obtained from the above-described processes are reduced to dryness to provide a solid concentrate of extracted plant phenolic compounds. In another embodiment, the resulting solid concentrate is further taken up into water to provide an aqueous solution having insoluble suspended material and this insoluble material is removed, for example by centrifuging or filtering, to provide a clear aqueous solution of extracted plant phenolic compounds.

In an embodiment of the application the stock solution of extracted plant phenolic compounds, the solid concentrate of extracted plant phenolic compounds or the aqueous solution of extracted plant phenolic compounds is treated to remove sugar compounds. In another embodiment the sugars are removed by chromatography, for example flash column chromatography. In another embodiment, the solid support or stationary phase in the column is a C₁₈ resin or any other support that absorbs hydrophobic compounds (for example, Amberlite XAD 16 or Sorbent SP207-05). The extracted plant phenolic compounds are typically removed from the column by flushing the column with a food-grade solvent and the resulting phenolic compound-containing solution is used as is or the solvent is removed to provide a solid concentrate of sugar-removed, extracted plant phenolic compounds. By “sugar-removed” it is meant that the sample is substantially sugar free. This solid concentrate is, again, used as is, freeze dried for storage or taken up in another solvent for use as a stock solution of extracted plant phenolic compounds.

In a further embodiment of the present application a food-grade carrier is added to the stock solution of extracted plant phenolic compounds, the solid concentrate of extracted plant phenolic compounds or the aqueous solution of extracted plant phenolic compounds or the essentially sugar-free versions thereof. Examples of such carriers include, but are not limited to maltodextrin, rice dextrin, modified starch and edible gums.

The present application also includes a product comprising an enhanced concentration of phenolics that have been extracted from fruits, particularly from apples, in accordance with the present application. In an embodiment, the product is in the form of emulsions, oils, liquids, solids, creams, or microencapsulated products. In another embodiment, the product is a animal or human food product. In another embodiment, the product is an oil-in-water emulsion, such as soups, salad dressings, and sauces, or a bulk oil, such as fish oil. In addition to the extended shelf life of polyunsaturated fatty acid- and/or lipid-containing foods and nutraceutical products, their health promoting properties can be enhanced by this novel antioxidant.

Although the apple skin extracts are very complex mixtures of compounds with differing antioxidant properties, it is interesting to note correlations between concentrations of antioxidant compounds and antioxidant capacity. The apple skin extracts of the new breed ‘KAR-27’ had a high concentration of phenolics (700 mg/100 g DW) measured by HPLC-MS/MS as well as relatively high antioxidant capacity measured by Folin Ciocalteu, FRAP, ORAC, and PUFA oxidation assays. The assays used in the present study measure antioxidant capacity by two different mechanisms, single electron transfer (SET) and hydrogen atom transfer (HAT). The Folin-Ciocalteu and FRAP assays are examples of assays that operate by SET while the ORAC assay operates by HAT (Prior and others 2005). Considering these mechanisms, it is interesting to note the correlations among the assays; the FRAP and Folin-Ciocalteu assays showed the highest degree of correlation, whereas the correlations among ORAC and FRAP and Folin-Ciocalteu were lower. For the methyl linolenate system the correlation coefficient was highest with the FRAP assay and lowest with the ORAC assay. However, the mechanism of antioxidant action, SET or HAT, is influenced by the pH of the reaction mixture (Lemanska and others 2001) and SET mechanics generally operate at low or high pH, whereas HAT at neutral pH (Prior and others 2005). Of all antioxidant capacity assays, the capacity determined by the FRAP assay was most strongly and similarly correlated with both the total phenolic concentration determined by HPLC-MS/MS and the antioxidant capacity for inhibition of the oxidation of methyl linolenate.

The methyl linolenate model system was used for determining the potential of apple skin extract as an inhibitor of oxidation for PUFA or omega-3 fatty acid containing food products of oil-in-water emulsions such as soups, salad dressings, and sauces. Inhibition of PUFA oxidation in this model system was moderately correlated with all of the antioxidant capacity measures thus showing that the antioxidant capacity assays, Folin-Ciocalteu, FRAP, and ORAC, could be used effectively in screening fruit extracts prior to their use in food model systems. The concentration of epicatechin in the apple extracts had the strongest correlation with the extract's ability to inhibit peroxyl radical-induced oxidation of methyl linolenate.

This is the first study to demonstrate the potential of apple skin extracts to prevent oxidation of omega-3 fatty acid.

The following non-limiting examples are illustrative of the present invention.

### EXAMPLES

### Example 1

### Phenolic Profiles and Antioxidant Properties of Apple Skin Extracts

### Materials and Chemicals

Apples were harvested at commercial maturity for commercial cultivars and at physiological maturity for new breeds and crab apples/heritage cultivar during the production years of 2005 and 2006 from the Atlantic Food and Horticultural Research Center of Agriculture and Agri-Food Canada (AFHRC-AAFC), Kentville, Nova Scotia, Canada. In the first sampling year, three different trees of each genotype were selected for the collection of the commercial cultivars. Where possible, apples were collected from three different trees for each of the new breeds and crab apple genotypes. In situations where only one tree of a particular breeding line or crab apple genotypes was available, three replicates were prepared by randomly grouping apples from the same tree. In the second sampling year three replicates were randomly collected from a pool of available apples including the situations where only the original tree was available.

HPLC-grade methanol, acetonitrile, and formic acid were purchased from Sigma-Aldrich (St. Louis, Mo., USA). The liquid chromatography standards used for the study were obtained as follows: quercetin-3-O-rhamnoside (quercitrin) and quercetin-3-O-galactoside (hyperin) were from Indofine Chemical Company (Hillsborough, N.J., USA); quercetin-3-O-glucoside (isoquercitrin), phloridzin, and chlorogenic acid were from Sigma-Aldrich (St. Louis, Mo., USA); quercetin-3-O-rutinoside (rutin), (−)-epicatechin, (+)-catechin, and procyanidin B1 and B2 were from ChromaDex (Santa Ana, Calif., USA); and cyanidin-3-O-galactoside was obtained from Extra-Synthase (Paris, France).

For the antioxidant capacity assays, the Folin-Ciocalteu reagent, gallic acid, 2,4,6-tris(2-pyridyl)-S-triazine (TPTZ), 6-hydroxy-2,5,7,8-tetramethylchroman-2-carboxylic acid (Trolox), and fluorescein sodium salt were purchased from Sigma-Aldrich (St. Louis, Mo., USA). The 2,2′-azobis(2-amidinopropane)dihydrochloride (AAPH) was acquired from Wako Chemicals (Richmond, Va., USA). The remaining chemicals were obtained from Fisher Scientific (Ottawa, ON, Canada).

### Extraction of Phenolic Compounds

The apples were washed and air-dried before they were peeled and cored using a bench-top apple peeler (Fox Run Craftsmen, Calif., USA). The skins and flesh were immediately frozen in liquid nitrogen and stored at −70° C. The frozen samples were lyophilized, using a freeze dryer (Model X3/SM-E, Edwards Vacuum, Mississauga, ON, Canada), and ground to a fine powder using a coffee grinder. Fifteen milliliters of methanol was used to extract the phenolics from 0.3 g of dehydrated apple tissue in 20 mL capacity amber glass vials. The mixtures were subjected to sonication (30 kHz; model 750D, VWR International Ltd., Montreal, QC, Canada) for 15 min. The crude extract was centrifuged at 3000 rpm for 15 min (model Durafuge™ 300, Precision Scientific, Asheville, N.C., USA) and an aliquot of the supernatant filtered through 0.2 μm nylon membrane in preparation for analysis by HPLC-MS/MS.

The extraction method used for the proanthocyanidins was adapted from Vidal and others (2003). The same lyophilized samples as used above were first washed with hexane (0.5 g of sample in 15 mL of hexane) to remove lipids, carotenoids, and chloropyhlls. The mixture was briefly subjected to sonication for 15 min×2 times, with a 10 min interval in between sonication cycles. The resulting mixture was filtered through 6 layers of cheesecloth. Fifteen milliliters of acetone:water:acetic acid (60:39:1 v/v) was added to the remaining solids. The resulting mixture was sonicated for 15 min×3 times, with 10 min intervals in between sonication cycles. The crude extract was centrifuged at 3000 rpm for 15 min and an aliquot of the supernatant filtered through 0.2 μm nylon membrane in preparation for analysis by HPLC-MS/MS.

### High Performance Liquid Chromatography and Mass Spectrometry Analysis

The HPLC system consisted of a Waters Alliance 2695 Separation Module that contained a quaternary pump and autosampler. The reverse phase column used was a Phenomenex Luna C₁₈ (150 mm×2.1 mm, 5 μm) with a Waters X-Terra MS C₁₈ guard column. A previously reported method (Rupasinghe and others 2008) was used for the analysis of flavan-3-ols, flavonols, dihydrochalcones, and phenolic acids. Briefly, a linear gradient elution was carried out with 0.1% formic acid in water (Solvent A) and 0.1% formic acid in acetonitrile (Solvent B) at a flow rate of 0.35 mL/min as follows: time t (min); (t, A %): (0, 94%), (9, 83.5%), (11.5, 83%), (14, 82.5%), (16, 82.5%), (18, 81.5%), (21, 80%), (29, 0%), (31, 94%), (40, 94%). Separation of the anthocyanin compounds was performed using the same HPLC system with 5% formic acid in water (A) and 5% formic acid in methanol (B) at a flow rate of 0.35 mL/min with the following linear gradient profile; (t, A %): (0.90%), (10, 70%), (17, 60%), (21, 48.8%), (26, 36%), (30, 10%), (31, 90%), (37, 90%). For the separation of the proanthocyanidins, the same HPLC system was used with a Phenomenex Luna C₁₈ (150 mm×4.6 mm, 5 μm) and a Waters X-Terra MS C₁₈ guard column. Ten microlitres of the proanthocyanidin fraction was injected onto the column using a method adapted from Friedrich and others (2000). The mobile phase consisted of a mixture of 0.2% acetic acid in water (A) and acetonitrile (B) (flow rate of 0.6 mL/min). A linear gradient elution during the first 30 minutes consisted of 95% A to 20% A, the mobile phase was then maintained at 20% A for 10 min. The system was returned to 95% A over 2 min, and held at 95% A for 8 min.

Electrospray ionization in negative ion mode (ESI−) was used for the analysis of the flavonol, flavan-3-ol, phenolic acid, dihydrochalcone, and procyanidin compounds. The following conditions were used: capillary voltage at 3000 V, temperature at 375° C., and the nebulizer gas (N₂) at a flow rate of 0.35 mL/min. For the analysis of the anthocyanin compounds, electrospray ionization in positive ion mode (ESI+) was used. The settings for the positive ion experiments were as follows: capillary voltage at 3500 V, temperature at 375° C., and the nebulizer gas (N₂) at a flow rate of 0.35 mL/min. The cone voltage (25-50 V) was optimized for each individual compound. Multiple reaction-monitoring (MRM) mode using specific precursor/product ion transitions was employed for quantification in comparison with standards (Table 1). In MRM experiments, both quadrupoles were operated at unit resolution.

### Folin-Ciocalteu Assay

Antioxidant capacity of the methanolic extracts was determined according to the Folin-Ciocalteu assay as described by Singleton and others (1999) with some modifications. Gallic acid was used for the generation of a standard curve using the extraction solvent (100% methanol) and diluted to 1.18, 2.35, 3.53, 4.70, 5.88, and 8.82 μM concentrations. The solutions were made fresh under reduced light conditions and the reaction was carried out under dark conditions. Twenty micro liters of the diluted extract, or gallic acid standard was mixed with 100 μL of 0.2 N Folin-Ciocalteu's phenol reagent in 96-well, clear, polystyrene microplates (COSTAR™ 9017) and gently mixed. After 6 min, 80 μL of 7.5% (w/v) sodium carbonate was added to each well and mixed. The mixture was incubated for 2 h at ambient temperature before absorption was measured at 760 nm using the FLUOstar OPTIMA™ plate reader (BMG Labtech, Durham, N.C., USA). Results were expressed as mg of gallic acid equivalent (GAE) per 100 g dry weight (mg GAE 100 g⁻¹ DW).

### The Ferric Reducing Antioxidant Power (FRAP) Assay

The FRAP assay was performed according to Benzie and Strain (1996) with some modifications. The reaction reagent (FRAP solution) was made immediately before the assay by mixing 300 mM acetate buffer (pH 3.6), 10 mM TPTZ solution, and 20 mM ferric chloride solution in the ratio of 10:1:1. The TPTZ solution was prepared the same day as the analysis. The Trolox standard solutions were prepared by diluting a 1 mM Trolox in methanol stock solution to make 5, 10, 25, 75, 150 and 300 μM Trolox concentrations in methanol. The FRAP analysis was performed by reacting 20 μL of blank, standard or sample with 180 μL FRAP solution in 96-well clear polystyrene plates (COSTAR 9017). The FLUOstar OPTIMA plate reader with an incubator and injection pump (BMG Labtech, Durham, N.C., USA) was programmed using the BMG Labtech software to take an absorbance reading at 595 nm, 6 min after the injection of the FRAP solution and a shaking time of 3 s. Both the FRAP solution and the samples in the microplate were warmed to 37° C. prior to assay. FRAP values were expressed as g Trolox equivalents (TE) per 100 g sample dry weight.

### Oxygen Radical Absorbance Capacity (ORAC) Assay

The hydrophilic ORAC assay (Cao and others 1993) as modified for a high through-put microplate reader (Huang and others 2002) was adapted for the laboratory as follows. The fluorescein sodium salt (0.957 μM) as well as samples and standards were dissolved in 75 mM phosphate buffer (K₂HPO₄/NaH₂PO₄, pH 7). Thirty-five microlitres of the sample or Trolox standard and 130 μL of the fluorescein probe were combined in the wells of the black 96-well polystyrene microplate (COSTAR 3915, Fisher Scientific, Ottawa, ON, Canada) and the plate was warmed to 37° C. for five minutes. The injection port was used to inject 35 μL 150 mM pre-warmed (37° C.) AAPH into the wells. The microplate was shaken for 3 s after injection of MPH and prior to each reading. The plate was maintained at 37° C. for the duration of the analyses (approximately 45 min) with excitation and emission readings every 80 seconds for the first two minutes then at every two minutes for the remaining 43 min. Excitation of the reaction mixture was at 490 nm and the emission was read at 510 nm. The antioxidant capacity of the samples was calculated as Trolox equivalents using a quadratic relation developed from area under the fluorescence decay curves for standards made to 5, 10, 25, 50, 75 μM concentrations, relative to the blank.

### Methyl Linolenate Model System and Thiobarbituric Acid Reactive Substances (TBARS) Assay

The apple skin samples from the second collection year, 2006, were examined for their potential to inhibit oxidation of PUFA using the oil-in-water emulsion system of methyl linolenate and the TBARS assay. The methyl linolenate model system and TBARS assay was adapted from methods reported by Okuda and others (2005) and Boadi and others (2003) for the laboratory as follows. Methyl linolenate (1.5 mg/mL) was suspended in a buffer solution (0.05 M TRIS-HCl, 0.15 M KCl, 1% Tween 20, pH 7.0) by homogenization for 30 s using a polytron homogenizer (Kinematica GmbH, Switzerland) and placed in 13×100 mm disposable glass tubes. Extracts (100 μL) diluted by 10-fold were added to the test tubes along with 100 μL of 0.1 M AAPH solution. The tubes were capped, vortexed, and placed in a shaker (150 rpm) (model Apollo HP50, CLP Tools, San Diego, Calif., USA) at room temperature for 26 hours.

For analysis using the TBARS assay, 100 μL of 2% BHT in ethanol was added to the test tubes and vortexed to stop the oxidation process. The thiobarbituric acid reagent (1 mL of 15% w/v trichloroacetic acid and 0.375% w/v thiobarbituric acid in 0.25 M HCl) was added, mixed, and the reaction mixture was placed in an 80° C. water bath for 15 min. The standards, made with 1,1,3,3-tetraethoxypropane, were prepared to 1, 5, 10, 50, and 100 μM concentrations and mixed with an equal part of the thiobarbituric reagent and placed in the water bath for 15 min. After 15 min, the samples and standards were cooled to room temperature and centrifuged for 10 min at 2000 rpm. The absorbance of the supernatant was then measured at 535 nm using 96-well clear polystyrene microplates (COSTAR 9017, Fisher Scientific, Ottawa, ON, Canada) in the FLUOstar OPTIMA plate reader (BMG Labtech, Durham, N.C., USA). The TBARS assay measures the dialdehyde compounds produced through the oxidation of methyl linolenate and the results are reported as percent inhibition of oxidation provided by the apple skin extracts. The maximum oxidation was determined using control samples that were exposed to oxidation without protection from antioxidants. Controls consisting of the apple extract in the Tris HCl buffer were made to determine potential contribution to absorbance at 535 nm from pigments in the apple extract (Hodges and others 1999). In most cases the absorbance contribution from the apple extract alone was lower than or equal to the blank, otherwise the additional absorbance was removed.

### Experimental Design and Statistical Analysis

The experimental design was a completely randomized block design with three replicates for the commercial cultivars, new breeding lines, and crab apples/heritage cultivar. SAS V8 (Cary, N.C., USA) was used for an analysis of variance, blocked by year, among the total phenolic values and among the phenolic profiles, α=0.05. Tukey's multiple means analysis was used to compare the values and assign letter groupings. One-way ANOVA, blocked by year, with Tukey comparisons was also used for ranking the genotypes based on antioxidant capacity assays. Comparisons of percent distribution for the phenolic compounds between years were examined using paired t-tests (MINITAB 14.1; State College, Pa., USA). Correlation analyses were performed using MINITAB 14.1 (State College, Pa., USA) with Pearson correlation coefficients recorded. Graphical representations were made using SigmaPlot 10.0 (San Jose, Calif., USA).

### Results

### Distribution of Phenolic Compounds Among Apple Genotypes

Since the majority of phenolics in apples are found in the skin, this study focused on evaluating the antioxidant properties of apple skin extracts. The phenolic compounds quantified in the apple skin were: the proanthocyanidins (procyanidin B1 and B2), the flavan-3-ols (epicatechin and catechin), the flavonols (quercetin-3-O-galactoside, quercetin-3-O-rhamnoside, quercetin-3-O-glucoside, quercetin-3-O-rutinoside), the dihydrochalcone (phloretin-2-O-glucoside), the anthocyanin (cyanidin-3-O-galactoside) and the phenolic acid (chlorogenic acid).

The concentrations of individual phenolic compounds varied greatly among tested apple genotypes with cyanidin-3-O-galactoside, on average, in highest quantity (124 mg/100 g DW) (Table 1). The mean distribution of phenolic compounds for all cultivars tested over two years of study (2005 and 2006) was as follows: 22% cyanidin-3-O-galactoside, 16% quercetin-3-O-galactoside, 12% quercetin-3-O-rhamnoside, 11% procyanidin B2, 11% epicatechin, 9% chlorogenic acid, 6% phloretin-2-O-glucoside, 5% quercetin-3-O-glucoside, 4% procyanidin B1, 3% quercetin-3-O-rutinoside, and 1% for catechin (Table 1).

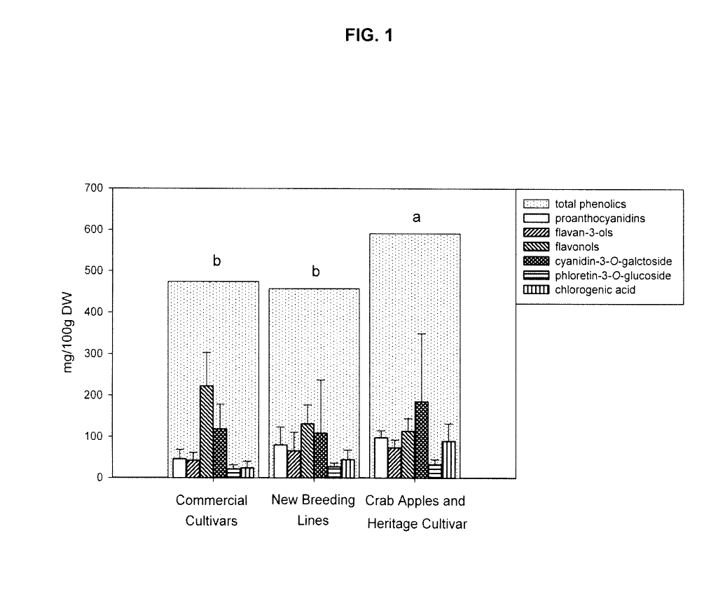

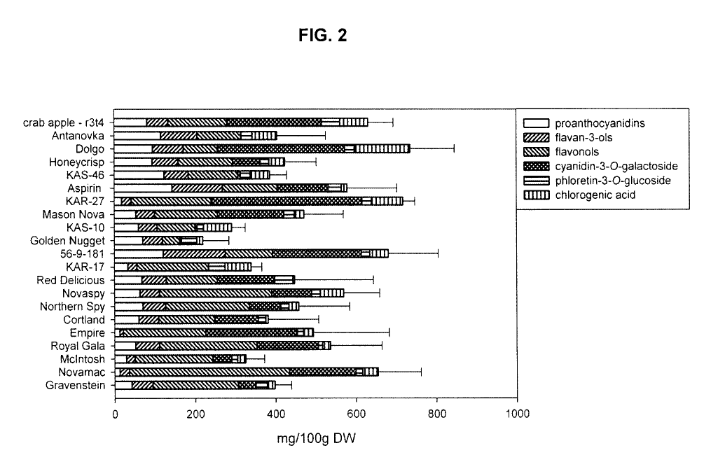

There were significant differences in phenolic profile among the genotypes (p<0.001) and trends in phenolic distribution among three groups of apple genotypes were examined (FIG. 1). Due to the great variation in concentrations of each phenolic compound examined, trends are not easily apparent from FIG. 1. It is interesting to note that total phenolics including the proanthocyanidin concentrations were higher for the crab apples/heritage cultivar group (p=0.013) and the year of analysis was not a significant factor (p=0.166) (FIG. 1). Additionally, the commercial cultivar group had higher concentrations of flavonols (p=0.010) and lower concentrations of proanthocyanidins and chlorogenic acid (p=0.042 and p=0.002, respectively) than new breeding lines and crab apples/heritage cultivar. While in contrast, the crab apples/heritage cultivar group had higher concentrations of chlorogenic acid than both the commercial cultivars and new breeding lines. The distribution of phenolics for each genotype over both collection years, 2005 and 2006, is shown in FIG. 2.

### Total Phenolics and Antioxidant Capacity of Apple Genotypes

The difference between harvest years was found to be significant for the Folin-Ciocalteu and FRAP antioxidant capacity assays (p<0.001), but not for the ORAC assay (p=0.215) (Table 2). The concentration of total phenolics in the methanolic extracts, measured using HPLC-MS/MS, also differed between 2005 and 2006 (p=0.002) (Table 2).

The range of phenolic concentrations in the methanolic extracts of the apple genotypes examined was from 150 to 700 mg per 100 g DW, which is more than a 4-fold difference. Among the skin extracts of all tested genotypes, a new breed that was developed at AFHRC-AAFC in Kentville, Nova Scotia, Canada, KAR-27, the crab apple species Dolgo', and the commercial cultivar, Novamac showed higher concentration of total phenolics for both 2005 and 2006 collections (FIG. 2; Table 2).

The antioxidant capacity measures; Folin-Ciocalteu (16.2 to 34.1 mg GAE/100 g DW), FRAP (1.3 to 3.3 g TE/100 g DW), and ORAC (5.2 to 14.2 g TE/100 g DW) had smaller ranges than the total phenolics in the methanol extracts, approximately 2-fold, 2.5-fold, and 3-fold, respectively (Table 2). The range of the % inhibition of peroxyl radical-induced oxidation of methyl linolenate was very small relative to the other antioxidant capacity assays (from 97.2% to 73.8%) (Table 2). The methanolic extracts of the skins of all the apple genotypes were effective inhibitors of peroxyl radical-induced oxidation of methyl linolenate as all the extracts showed over 73% inhibition (Table 2). Among the 21 apple skin extracts tested, those prepared from ‘Antanovka’ (the old Russian heritage cultivar) and crab apple Dolgo', the new breeding lines ‘KAS-46’, ‘Aspirin’, and ‘56-9-181’, and the commercial cultivar ‘Royal Gala’ were the most effective in the inhibition of oxidation of methyl linolenate (Table 2).

### Correlation Analysis Between Phenolic Compounds and Antioxidant Measures

Pearson correlation analyses showed that among the phenolic compounds identified in the methanolic extracts (−)-epicatechin, (−)-catechin, cyanidin-3-O-galactoside, phloridzin, and chlorogenic acid were correlated with the antioxidant capacity assays. However, the concentrations of flavonols were not correlated or negatively correlated with the antioxidant capacity assays (Table 3). Significant linear relationships between the antioxidant capacity assays were shown, stronger between Folin-Ciocalteu and FRAP assays and Folin-Ciocalteu and ORAC assays, and less pronounced between the FRAP and ORAC assays (Table 3). Correlations between the antioxidant capacity exhibited between the methyl linolenate oxidation system and different antioxidant capacity measures were significant and strongest with the FRAP assay. The antioxidant capacity measures Folin-Ciocalteu, FRAP, ORAC and the methyl linolenate model system were correlated with total phenolics measured by HPLC-MS/MS, α=0.05 (Table 3).

### Conclusions

Apple skin, a by-product of apple processing, was found as a potential source of natural antioxidants. The concentration of total phenolic compound present in methanolic extracts ranged from 150 to 700 mg/100 g DW among 21 genotypes evaluated. The total antioxidant capacity of the apple skin extracts was also highly varied: Folin-Ciocalteu (16.2 to 34.1 mg GAE/100 g DW), FRAP (1.3 to 3.3 g TE/100 g DW) and ORAC (5.2 to 14.2 g TE/100 g DW). Interestingly, the apple skin extracts were found to possess strong lipid stabilizing ability of 73.8% to 97.2% inhibition of peroxyl radical-mediated oxidation of methyl linolenate in an aqueous emulsion system. The apple skin extracts of crab apples were shown to be effective inhibitors of lipid oxidation indicating a potential use for this underutilized bio-resource in the development of natural food antioxidants.

### Example 2

### Apple Skin Extracts to Reduce Lipid Oxidation

### Materials and Methods

### Chemicals

Omega-3 fatty acids ML, EPA and DHA were obtained from Nu-Chek Prep, Inc. (Elysian, Minn., USA) and the fish oil [03/55 TG fish oil, CFIA reg. 3529; 61% EPA, 4.3% DHA, 17.6 monounsaturated, 77.6 polyunsaturated fatty acid by weight of total fatty acids] was a generous gift from Ocean Nutrition Canada, Dartmouth, NS, Canada. Butylated hydroxyl toluene (BHT), tert-butyl hydroquinone (TBHQ), -tocopherol, 2,2-azobis(2-amidinopropane), 1,1,3,3-tetraethoxypropane (TEP), ferrous sulfate (FeSO₄), ammoniumthiocyanate (NH₄SCN), isooctane and trichloroacetic acid (TCA) were obtained from Sigma-Aldrich Canada. Lipid hydroperoxide standard, 13-hydroperoxyoctadecanoic acid (13-HpODE) was obtained from MP Biomedicals, Canada. The 96-well microplates were purchased from Fisher Scientific (Ottawa, ON, Canada). All other chemicals and reagent were purchased from Fisher Scientific, Canada with the highest grade in their purity.

### Preparation of Extracts

Apple skin extract 1 was prepared by two methods: directly using freshly peeled apple skins or using dehydrated apple skin powder. Apple fruit skins (thickness of 1 to 2 mm) of ‘Northern Spy’ were collected from a commercial pie manufacturer, Apple Valley Foods Inc., Kentville, NS, Canada. Immediately after peeling (preferably within 10 min), the skins were submerged in a solution of 2% (w/v) calcium chloride (CaCl₂) in water at 60±5° C. for 10 min to preserve the antioxidant compounds presence in apple skins. After draining the excess water and within 3 h of blanching treatment, the CaCl₂-treated apple skins were transported in plastic containers to the Nova Scotia Agricultural College (NSAC). The CaCl₂-treated apple skins were either ground in to a slurry using an Ursher Mill (or equivalent equipment) or used immediately or freezed for later use. The frozen apple skins can be ground using an Ursher MIII or equivalent equipment and used for the next step directly. The apple skin slurry was extracted with ethanol (preferably the ratio of 1 kg of slurry to 4 L of ethanol; preferably 100% ethanol but 40% to 100% ethanol can also be used). However, other solvents can be used. The extraction process was assisted by using an ultrasonication bath (20 kHz) for 30 min; however, other techniques to facilitate the extraction can also be used. The solids were then separated from the liquid using either centrifugation (3000 rpm for 10 min) or using a fruit press followed by a vacuum filtration in a buchner funnel with a Whatman P8 filter paper. The resulted liquid was evaporated using a rotary evaporator under reduced pressure at 35° C. The resulted concentrated liquid extract was filtered as above using a buchner funnel with a Whatman P8 filter paper and used directly as apple skin extract 1 or for preparation of apple skin extract 2 after removal of sugars from antioxidants, mainly phenolic compounds.

As an alternative method of preparation for apple skin extract 1, dehydrated apple skin powder was prepared from the above mentioned CaCl₂-treated apple skins. The apple skins were dried in wire-meshed trays at 60±5° C. for 48 h using a convection oven with air circulation (Milner Agincourt, ON, Canada). The dried skins were ground into a fine powder using a Willey mill with 1 mm screen (Arthur Thomas Co., Philadelphia, Pa., USA). Ten grams of dehydrated apple skin powder was weighed into a conical flask and added 100 ml of 95% ethanol. The suspension was stirred gently and subjected to sonication two times for 15 min with 10 min intervals in between sonication cycles. The suspension was then transferred into a 50 ml corning tube for centrifugation at 3000 rpm for 15 min. The supernatants (apple skin extract 1) were collected in amber vial and stored at −20° C. until uses.

The apple skin extract 2 was prepared by using the method mentioned above for the apple skin extract 1. First, removal of sugar from apple skin extract 1 was performed by flash chromatography using a C₁₈ or any other resin (e.g. Amberlite XAD 16, Sorbent SP207-05) that can absorb hydrophobic compounds. In contrast, normal phase flash chromatography can also be performed to separate sugars from phenolic compounds of the apple skin extract 1. For example, Amberlite XAD 16 column was conditioned with water and then the concentrated apple skin extract 1 was loaded at the top of the column slowly. Once the column was loaded with concentrated apple skin extract, the column was washed with water by sending 2 to 3 times of bed volume of water through the column. The removal of sugar was monitored by measuring the Brix value of wash water using a refractrometer. Once the Brix value was less than 1%, washing step was terminated. The phenolic antioxidants retained in the column were eluted using 100% ethanol and the elute was concentrated using a rotary evaporator at 35° C. The sugar removed concentrated apple skin extract was then freeze dried to produce a powder, which can be stored under dark at freezing temperatures until use. In order to prepare apple skin extract 2, the sugar removed apple skin extract (powder form) was dissolved in ethanol in 1 g:2 ml ratio.

### Preparation of Aqueous Emulsions and Bulk Oil

To prepare aqueous emulsion for these substrates a modified method was followed based on Okuda et al. (2005) and Boadi et al. (2003). Briefly, the emulsion of each substrate was prepared at the concentration of 1.5 mg substrate per mL of buffer as emulsifier containing 0.05 M Tris-HCl, 0.15 M KCl and 1% Tween 20 (pH 7) at room temperature. The sample was homogenized using a Polytron homogenizer (model PCU Drehzahlregler, Switzerland) at 4.5 speed for 30 s. The apple skin extracts or antioxidants were incorporated in emulsions by placing specific volumes of stock solutions to obtain desirable final concentration in each test tube. The solvent (ethanol) of the added extracts or antioxidants were removed completely under nitrogen and then mixed with 0.9 mL (for peroxyl radical-induced oxidation) or 1 mL (for heat- and UV-induced oxidation) of the emulsion in disposable borosilicate glass tubes (13×100 mm). The resulting emulsions were also made to contain 10% ethanol in order to ensure the complete dissolution of extract.

The bulk fish oil model system was created by oxidizing 100 μL of the fish oil in 13×100 mm borosilicate glass tubes with caps. The apple skin extracts or antioxidants were incorporated by placing desirable volumes in each test tube, drying the solvent completely under nitrogen, and then mixing with 100 μL of the fish oil. To ensure the complete dissolution of extracts and antioxidants, 20% ethanol was used.

### Induction of Oxidation

Oxidation conditions for emulsions and bulk oil samples were optimized separately to provide maximum hydroperoxide formation for ferric thiocyanate and maximum secondary oxidation products for thiobarbituric acid reactive substances (TBARS) assay. For the measurement of TBARS for the emulsion of ML, EPA, and DHA with or without apple skin extracts or antioxidants, the following three different methods of induction of oxidation were used: (i.) heating at 70° C. for three hours using a shaking water bath, (ii.) adding peroxyl radical generator, AAPH (100 μl of 100 mM) to the emulsions at room temperature and maintained at room temperature for 24 h using a horizontally rotating shaker at 150 rpm, and (iii.) exposing the emulsions to UV at room temperature (one Full Spectrum Terrarium Lamp at 18 cm distance, Repti Glo 2.0 uvB; 800 Lumen, 13 Watt, HAGEN, China) for 24 h using a horizontally rotating shaker at 150 rpm. For the measurement of primary oxidation products of DHA emulsions by ferric thiocyanate (FTC) assay the methods of induction of oxidation were: heating at 70° C. for 2 min as above, (ii.) exposing to the UV light at room temperature for 20 min. For the TBARS measurements, the induction conditions for bulk oil were similar to the conditions mentioned above for emulsions. For the FTC assay of bulk oil samples, heating at 70° C. for 10 min and exposure to UV light at room temperature for 1 h were the optimum conditions. At the end of oxidations, 100 μL and 10 μL of 1000 ppm BHT in ethanol were added to emulsion and bulk oil samples, respectively, to stop oxidation immediately. Oxidized samples have been kept in deep freeze (−20° C.) until analysis. Triplicate samples were subjected to oxidation upon each concentration and performed with appropriate controls (no antioxidant-no induction and no antioxidant-with induction), and all the experiments were conducted independently twice.

### Ferric Thiocyanate Test

The procedure was adapted from Osawa and Namiki (1981) to perform in a 96-FLUOstar OPTIMA microplate reader (BMG labtech, Durham, N.C., USA) as follows: At the end of the incubation of emulsion or bulk oil samples, 30 μL aliquot was taken from the mixture and diluted with 210 μL of 75% ethanol, followed by the addition of 30 μL of 3% ammonium thiocyanate (NH₄SCN). Precisely 3 min after adding 30 μL of 2 mM ferric chloride (FeCl₂) in 3.5% HCl, the absorbance for the red color was measured at 500 nm. The level of lipid oxidation in all oxidized samples was calculated as percent inhibition according to the following equation:

% Inhibition of oxidation=(A_{control}−Aₛₐₘₚₗₑ)/A_{control}×100 (I)

wherein, Aₛₐₘₚₗₑ represents the absorbance for the sample containing the antioxidant and A_{control} represents the absorbance for the sample that does not contain any antioxidants. Ferric thiocyanate test was performed in triplicate for all samples in two set of experiments at different times.

### Thiobarbituric Acid Reactive Substances (TBARS) Assay

After the completion of the oxidation treatment, TBARS were quantified by a modified method of Boadi et al. (2003) and Okuda et al. (2005), as follows. One-hundred microliters of 2% BHT in ethanol were added to the test tubes to stop the oxidation process. The TBA reagent (1 mL of 15% (w/v) trichloroacetic acid and 0.375% (w/v) TBA in 0.25 M HCl) was then added and mixed. The reaction mixture was placed in a water bath at 80° C. for 15 min. The standards, made with 1,1,3,3-tetraethoxypropane (TEP), were prepared at 1, 5, 10, 50, and 100 μM concentrations and mixed with an equal portion of the TBA reagent and were also placed in the water bath (80° C.) for 15 min. After 15 min, the samples and standards were cooled to room temperature and centrifuged at 2000 rpm for 15 min (model Durafuge 300, Precision Scientific, Asheville, N.C., USA). The absorbance of the supernatant was then measured at 532 nm using 96-well microplates in the FLUOstar OPTIMA plate reader (BMG Labtech, Durham, N.C., USA). The outer wells of the microplates were not included to ensure temperature uniformity in all wells. After the subtraction of blank values, absorbance values were converted to mg malondialdehyde (MDA) equivalents per mg of PUFA substrate using the standard curve developed for each experiment. Percent inhibition of oxidation was calculated as a percentage of the total oxidation experienced by the system without the protection of antioxidants using the formulation in equation (I) above.

### Evaluation of Oxidative Stability Under Accelerated Conditions Using Rancimat

The resistance to auto-oxidation was measured using the Rancimat 743 (Metrohm AG, Herisau, Switzerland) instrument at 70, 90, 100 and 110° C. with the air flow rate of 20 Uh. Five concentrations of apple skin extract 1 and apple skin extract 2 were incorporated in to 3.0±0.1 g of fish oil samples and oxidative stability was determined based on the induction time (IT) at 100° C. The concentrations of the apple skin extracts with the highest oxidative stability were evaluated further at 70, 90 and 110° C. for the estimation of storage time of fish oil at room temperature. All the experiments were done in triplicate.

### Total Phenols and Antioxidant Capacity Assays

Determinations of total phenolic content using Folin-Ciocalteu assay and total antioxidant capacity using the oxygen radical absorbance capacity (ORAC) assay and the ferric reducing antioxidant power (FRAP) assay were performed using the methods reported by Rupasinghe et al. (2008). DPPH assay was performed using the procedure described by Frankel et al., (2000).

### Liquid Chromatography Mass Spectrometry Analysis of Phenolics

Analyses of major individual phenolic compounds present in apple skin extracts were performed using Rupasinghe et al. (2008).

### Statistical Analysis

The all measurements were done in triplicate and the values are reported as mean along with standard deviation (SD). To determine the significance of difference among the mean value one-way analysis of variance (ANOVA) followed by Tukey's honestly significant difference (HSD) multiple comparisons were performed at a level of P<0.05 using SAS 9.1 statistical software.

### Results

### Phenolic Constituents in Apple Skin Extracts

The total of major phenolics present in apple skin extract 1 and 2 determined by using HPLC-ES-MS/MS were 399 μg/mL and 42025 μg/mL, respectively (Table 4). The major polyphenolic compounds detected from apple skins belong to sub-classes of flavonols, dihydrochalcones, flavan-3-ols, phenolic acids, and anthocyanins (Table 4).

### Antioxidant Capacity of Apple Skin Extracts

The total antioxidant capacity measured using FRAP, ORAC, and DPPH assays indicated that apple skin extract 2 has several fold greater antioxidant capacity than apple skin extract 1 (Table 5). The IC₅₀ values measured by DPPH assay were obtained utilizing a calibration curve prepared by plotting percent inhibition values as a function of concentration of the test material. Each data point in the calibration curves are the mean of three repetitive determinations. Trolox was used as a positive control for comparing its radical scavenging activity with those of apple skin extracts. It can be clearly seen from IC₅₀ values that the apple skin extract 2 has the highest radical scavenging capacity represented by the lowest IC₅₀ value, followed by apple skin extract 1 and Trolox. Both apple skin extracts show higher antioxidant activity against the synthetic DPPH radical than Trolox which is the water soluble form of α-tocopherol. The magnitude of the differences seems to be dependent on the mode of action of the antioxidant capacity assays; however, the results agree with the content of total phenolics present in the extracts indicating that the phenolics are the key antioxidants present in these extracts.

### Inhibition of Lipid Oxidation in PUFA Emulsions

### (a) Emulsions—TBARS Results

Initially, the two apple skin extracts at 5 concentrations were evaluated for their ability to inhibit the heat-, peroxyl radical-, and UV-induced oxidation of ML, EPA, DHA in oil-in-water emulsions using the TBARS assay (Tables 6-8). The time for TBARS measurement was determined by preliminary experiments that were performed to observe the time-dependent TBARS formation by PUFA without any antioxidants after oxidation-induction by different methods (data not presented). The percent inhibition of oxidation was also compared to the selected concentration of three food antioxidants, α-tocopherol, BHT, and TBHQ.

In general, concentration dependent inhibition of formation of secondary products of PUFA oxidation was observed for apple skin extracts under all the three oxidation induction methods except at the very high concentrations of the extracts that the % inhibition tends to decrease (Table 6-8). Under the present experimental conditions, the ability of apple skin extracts to protect PUFA from UV-induced oxidation was relatively lesser than that of heat- and peroxyl radical-induced oxidation.

It was found that antioxidant properties of apple skin extract 2 are greater than that of apple skin extract 1 in term of total phenolic concentration (Table 6-8). This suggests that removal of sugars from the extract 1 has significantly increased the ability of polyphenolics to inhibit the lipid oxidation. It was interesting to note that α-tocopherol, was not an effective antioxidant in PUFA emulsions when compared to apple skin extracts or BHT and TBHQ.

### (b) Emulsions—Ferric Thiocyanate Test Results

Percent inhibition calculated based on the formation of primary oxidation products (lipid hydroperoxides) of heat- and UV-induced oxidation of DHA emulsions incorporated with apple skin extracts or food antioxidants is presented in Table 9. The results indicate that the suppression of the formation lipid hydroperoxides in DHA emulsions by apple skin extracts is concentration dependent (Table 9). It is also confirmed that apple skin extracts are effective in protection of PUFA against both of heat and UV exposure. When the concentration of total phenolics of the extracts is considered, it seems that apple skin extract 2 is more effective than apple skin extract 1 in terms of suppression of the formation of primary lipid oxidation products. When compared to BHT and TBHQ, α-tocopherol seems to be the weakest antioxidant for preserving DHA emulsions. Therefore, based on the results of secondary oxidation products (TBARS) measurement and primary oxidation products by ferric thiocyanate assay, α-tocopherol at 400 μg/mL seems to be a relatively less effective antioxidant to protect PUFA emulsions from oxidation.

### Inhibition of Lipid Oxidation in Bulk Fish Oil

### (a) Bulk Oil—TBARS Results

Interestingly, under the experimental conditions studied, almost complete inhibition of heat-, peroxyl radical-, and UV-induced oxidation of bulk fish oil was observed when apple skin extract 2 was incorporated in fish oil at the concentration of approximately 400 μg/mL of total phenolics (Table 10). The antioxidant properties of the above concentration of apple skin extract 1 seem to be comparable to that of α-tocopherol and BHT at the similar concentration. Thus, based on TBARS results, 400 μg/mL of total phenolics of the extract 2 could be the optimal concentration against formation of secondary oxidation products in bulk fish oil. Food antioxidants, α-tocopherol and BHT at the tested concentrations seem to be equally effective in preventing the oxidation of bulk fish oil under the three oxidation induction systems studied.

### (b) Bulk Oil—Ferric Thiocyanate Test Results

The increasing concentrations of apple skin extract 1 and 2 in bulk fish oil provided an increasing protection against heat- and UV-induced lipid oxidation (Table 11). However, when compared to the effectiveness of apple skin extracts in PUFA emulsions, the extracts were required in higher concentrations to obtain the similar effectiveness in bulk oil (Table 9 vs. 11). While not wishing to be limited by theory, this can be explained by the fact that higher amounts of hydroperoxides form in bulk oils than in emulsions because of the relative higher amount of fatty acids in a bulk oil system than in an emulsion system where the percent of lipid content is less than in a bulk lipid system. In contrast, the effectiveness of α-tocopherol seems to be greater in bulk oil than in emulsion in agreement with the polar paradox (Frankel, 1996). The chemical nature of α-tocopherol molecule due to its higher number of carbon atoms and an aliphatic side chain have provided a more non-polar and lipohilic character which can make it to dissolve it in the bulk oil phase more homogenously, providing a greater accessible to lipid peroxides formed during lipid oxidation.

### Accelerated Oxidation Test Using Rancimat

The accelerated oxidative test using Rancimat has been extensively used by researchers and industry to determine oxidative stability of lipids. The efficacy of antioxidants in lipids can be determined by the Induction time (IT), which is the time that elapses until the secondary oxidation products form under accelerated oxidation created by heat with the presence of a constant flow of air. In the present study, IT was determined in bulk fish oil when incorporated with apple skin extract with comparison to the food antioxidants, α-tocopherol and BHT. When IT values were determined at 100° C. for varying concentrations of apple skin extracts and α-tocopherol and BHT, a concentration dependent increase in IT was observed (Table 12). As well, the effectiveness of apple skins based on the total concentration of polyphenolics is equivalent to that of α-tocopherol.

The concentration of polyphenolics at approximately 400 μg/mL in apple skin extracts and 400 μg/mL of α-tocopherol were used to determine the IT at 70, 90 and 110° C. as well using the Rancimat for the estimation of storage time at room temperature (Table 13). The IT values for all the oil samples with and without incorporation of apple skin extracts, α-tocopherol and BHT are conversely proportional to the temperature (Table 10). The IT values of fish oil incorporated with apple skin extract at all the tested temperature were greater than the controls (fish oil without any antioxidants) at the respective temperatures (Table 13). As well, the estimated IT of the fish oil incorporated with the apple skin extract 2 at room temperature was similar to that of α-tocopherol (Table 14). This further solidifies that results obtained from TBARS and ferric thiocyanates test of heat-induced oxidation of fish oil that the apple skin extracts provide protection against lipid oxidation in fish oil. It is also interesting to note that the IT values of fish oil incorporated with apple skin extracts at 70° C. are significantly greater than that of the IT values at 90, 100 and 110° C. This large difference between IT values could be due to the fact that a high percent of fish oil is already decomposed at temperatures at and above 90° C.

In general, the apple skin extracts studied exhibited similar antioxidant properties under the accelerated oxidation conditions to that of α-tocopherol in the bulk oil phase, which is also confirmed with the results obtained from the TBARS and ferric thiocyanate tests. When the apple skin extracts were incorporated at very high concentrations of polyphenolics, the effectiveness seems to be less, probably due to the less solubility of the polyphenolics in the oil at high concentrations. The extract at this concentration could also be pro-oxidant, rather than antioxidant, when used in a range out of a particular range that is best for antioxidation in the oil.

Antioxidant activity of apple has been well studied and also especially apple skins have not only higher capacity in scavenging oxygen radical and total antioxidant activity but also greater concentrations of phenolic compounds than the flesh (Eberhardt et al., 2000; Wolfe et al., 2003; Vinson et al., 2001; Drogoudi et al. 2008). However, this is the first report to reveal that apple peel extracts possess strong ability to prevent oxidation of PUFA and PUFA containing oil.

### Conclusion

The present results reveal that apple skin extracts are strong natural antioxidants against oxidation of free PUFA in emulsions and PUFA containing bulk fish oil. The results conclusively demonstrate that apple skin extracts (≧2 ppm total phenolics) are capable of preserving or inhibiting the decomposition of PUFA and PUFA containing lipids against heat-, peroxyl radical- and UV-induced oxidation. Removal of sugar from the ethanolic extracts of apple skins has increased the capacity of polyphenolic antioxidants present in apple skin extract to stabilize PUFA and PUFA containing fish oil.

While the present application has been described with reference to what are presently considered to be the preferred examples, it is to be understood that the application is not limited to the disclosed examples. To the contrary, the application is intended to cover various modifications and equivalent arrangements included within the spirit and scope of the appended claims.

TABLE 1
Mean concentration (mg/100 g DW ± standard deviation; two harvest seasons),
percent distribution of phenolic compounds found in apple skin of 21 apple genotypes.

| | Retention | Parent Ion | Product Ion | Concentration | Distribution | 2005 vs 2006 |
|---|---|---|---|---|---|---|
| Phenolic Compound | Time (min) | (m/z) | (m/z) | (mg/100 g DW) | (%) | Harvest seasons |
| Proanthocyanidin B1 | 23.23 | 578 | 289 | 17 ± 11 | 4 | ** |
| Proanthocyanidin B2 | 26.07 | 578 | 289 | 51 ± 28 | 11 | NS |
| (−)-Epicatechin | 12.19 | 289 | 109 | 50 ± 27 | 11 | NS |
| (+)-Catechin | 9.85 | 289 | 109 | 7 ± 9 | 1 | NS |
| Quercetin-3-O-galactoside | 17.71 | 463 | 301 | 73 ± 34 | 16 | * |
| Quercetin-3-O- | 23.47 | 447 | 301 | 59 ± 42 | 12 | *** |
| rhamnoside | | | | | | |
| Quercetin-3-O-glucoside | 18.67 | 463 | 301 | 23 ± 18 | 5 | NS |
| Quercetin-3-O-rutinoside | 17.33 | 609 | 301 | 12 ± 10 | 3 | ** |
| Cyanidin-3-O-galactoside | 10.93 | 449 | 287 | 124 ± 107 | 22 | *** |
| Phloretin-2-O-glucoside | 26.41 | 435 | 273 | 26 ± 10 | 6 | *** |
| Chlorogenic acid | 10.05 | 353 | 191 | 42 ± 32 | 9 | NS |

NS represents no significant difference in compound distributions between the collection years p ≧ 0.05, whereas *** p ≦ 0.001, ** p ≦0.01, * p ≦ 0.05.

TABLE 2
Total phenolic concentration, the antioxidant capacity (Folin-Ciocalteu, FRAP, ORAC assays)
and percent inhibition of oxidation of methyl linolenate of the methanolic extracts of apple skin.

| | | Total Phenolic by | Folin Ciocalteu | FRAP | ORAC | % Inhibition of |
|---|---|---|---|---|---|---|
| Apple | | HPLC-MS/MS | (mgGAE/100 g | (gTE/100 g | (gTE/100 g | Methyl Linolenate |
| Genotype | | (mg/100 g DW)^{X} | DW) | DW) | DW) | Oxidation^{Y} |
| Crab Apples | crabapple - r3t4 | 551^{abcde} | 31.8^{ab} | 1.94^{bcde} | 13.4^{abc} | 94.5^{abc} |
| and Heritage | Antanovka | 290^{fghi} | 21.4^{def} | 1.25^{e} | 10.3^{abcde} | 96.5^{a} |
| Cultivar | Dolgo | 642^{ab} | 34.1^{a} | 3.28^{a} | 14.2^{a} | 95.7^{a} |
| New Breeding | Honeycrisp ™ | 323^{efghi} | 22.4^{def} | 1.99^{bcde} | 11.7^{abcd} | 95.4^{ab} |
| Lines | KAS-46 | 264^{ghi} | 26.6^{abcd} | 2.21^{bcd} | 14.6^{a} | 95.8^{a} |
| | Aspirin | 436^{cdefg} | 26.0^{bcd} | 2.58^{ab} | 13.3^{ab} | 97.2^{a} |
| | KAR-27 | 700^{a} | 24.1^{bcde} | 2.61^{ab} | 11.2^{abcde} | 93.8^{abc} |
| | Mason Nova | 419^{cdefgh} | 21.2^{def} | 1.41^{de} | 8.06^{abcde} | 94.3^{abc} |
| | KAS-10 | 232^{hi} | 19.4^{def} | 1.52^{de} | 11.0^{abcde} | 85.9^{d} |
| | Golden Nugget | 150ⁱ | 20.8^{def} | 1.31^{de} | 6.40^{cde} | 91.3^{abcd} |
| | 56-9-181 | 561^{abc} | 30.3^{abc} | 2.50^{abc} | 10.3^{abcde} | 96.5^{a} |
| | KAR-17 | 307^{efghi} | 21.0^{def} | 1.25^{e} | 5.37^{e} | 73.8^{e} |
| Commercial | Red Delicious | 380^{cdefgh} | 25.3^{bcd} | 2.17^{bcde} | 12.5^{abcd} | 91.7^{abcd} |
| Cultivars | Novaspy | 508^{bcdef} | 24^{cde} | 2.20^{bcd} | 11.4^{abcd} | 92.1^{abcd} |
| | Northern Spy | 405^{cdefgh} | 23.2^{cde} | 1.43^{de} | 10.8^{abcde} | 90.7^{abcd} |
| | Cortland | 322^{efghi} | 19.3^{def} | 1.73^{bcde} | 8.69^{abcde} | 89.1^{bcd} |
| | Empire | 482^{bcdef} | 15.3^{f} | 1.43^{de} | 6.77^{bcde} | 77.4^{e} |
| | Royal Gala | 484^{bcdef} | 22.4^{def} | 1.74^{bcde} | 13.5^{a} | 95.7^{a} |
| | McIntosh | 296^{fghi} | 17.2^{ef} | 1.33^{de} | 6.25^{de} | 88.8^{cd} |
| | Novamac | 642^{ab} | 21.1^{def} | 1.87^{bcde} | 11.2^{abcd} | 86.6^{d} |
| | Gravenstein | 356^{defgh} | 19.6^{def} | 1.61^{cde} | 8.45^{abcde} | 94.1^{abc} |
| | ANOVA | | | | | |
| | Year (blocking factor) | 0.002 | <0.001 | <0.001 | 0.083 | N/A^{z} |
| | Genotype (factor of | <0.001 | <0.001 | <0.001 | <0.001 | <0.001 |
| | interest) | | | | | |

Cultivar means were compared within column and means followed by same letter are not significantly different (α = 0.05).
^{X}Total phenolic concentration of methanolic extracts measured using HPLC-MS/MS
^{Y}Percentage inhibition of oxidation is expressed relative to the oxidation occurring in the absence of the extracts.
^{z}Measurements were done only for 2006 harvest season

TABLE 3
Pearson Correlation coefficients to show linear relationship among
the antioxidant capacity measures, Folin-Ciocalteu, FRAP, ORAC,
and the % inhibition of oxidations of methyl linolenate, and the phenolic
compounds^{z} found in the methanolic extracts of apple skin.

| | Total | | | | |
|---|---|---|---|---|---|
| | Phenolics | | | | Methyl |
| | by HPLC- | Folin | | | Linolenate |
| Compounds | MS/MS | Ciocalteu | FRAP | ORAC | Oxidation |
| (−)-Epicatechin | 0.16 | 0.52* | 0.36* | 0.53* | 0.56* |
| (−)-Catechin | 0.14 | 0.31* | 0.32* | 0.27* | 0.43* |
| Total Flavan-3- | 0.16 | 0.50* | 0.37* | 0.50* | 0.55* |
| ols | | | | | |
| Quercetin-3-O- | 0.45* | −0.05 | 0.09 | −0.06 | −0.10 |
| rhamnoside | | | | | |
| Quercetin-3-O- | 0.27 | −0.26* | −0.13 | −0.12 | −0.23 |
| glucoside | | | | | |
| Quercetin-3-O- | 0.22 | −0.14 | −0.09 | 0.18 | 0.09 |
| galactoside | | | | | |
| Quercetin-3-O- | 0.35* | −0.19* | −0.08 | 0.00 | 0.03 |
| rutinoside | | | | | |
| Total Flavonols | 0.44* | −0.17 | −0.04 | 0.02 | −0.06 |
| Cyanidin-3-O- | 0.85* | 0.45* | 0.64* | 0.15 | 0.28* |
| galactoside | | | | | |
| Phloridzin | 0.12 | 0.51* | 0.31* | 0.29* | 0.24 |
| Chlorogenic Acid | 0.36* | 0.40* | 0.34* | 0.23* | 0.12 |
| Total Phenolic by | — | 0.51* | 0.68* | 0.33* | 0.41* |
| HPLC-MS/MS | | | | | |
| Folin Ciocalteu | | — | 0.73* | 0.60* | 0.62* |
| FRAP | | | — | 0.33* | 0.66* |
| ORAC | | | | — | 0.51* |
| Methyl | | | | | — |
| Linolenate | | | | | |
| Oxidation | | | | | |

Correlation analyses involving the methyl linolenate model system include only second year results.
*Significant correlations are shown (p ≦ 0.05).
^{z}Proanthocyanidins were not considered for the correlation analysis since the extraction solvent is different from one used for all other phenolics and antioxidant assays.

TABLE 4
The concentration of polyphenolic compounds of the two apple skin
extracts prepared from ‘Northern Spy’ apples.

| | | Polyphenolic content¹ | |
|---|---|---|---|
| | | (μg/mL extract) | |
| Polyphenols | | Apple Skin | Apple Skin |
| category | Compound | Extract 1 | Extract 2 |
| Flavonols | Quercetin-3-O- | ND | 73.5 ± 4.1 |
| | peltoside | | |
| | Quercetin-3-O- | 133 ± 6.9 | 11774 ± 518 |
| | galactoside | | |
| | Quercetin-3-O- | 18.8 ± 1.2 | 3904 ± 156 |
| | glucoside | | |
| | Quercetin-3-O- | 53.0 ± 2.7 | 10440 ± 417 |
| | rhamnoside | | |
| | Quercetin-3-O- | 5.1 ± 0.4 | 2748 ± 104 |
| | rutinoside | | |
| | Quercetin | 2.2 ± 0.9 | 284 ± 11.4 |
| Dihydrochalcones | Phloridzin | 61.4 ± 2.9 | 1827 ± 71 |
| | Phloretin | 1.1 ± 0.1 | 9.5 ± 0.4 |
| Phenolic acids | Chlorogenic acid | 34.8 ± 2.4 | 4087 ± 204 |
| | Cafeic acid | 0.8 ± 0.08 | 36.5 ± 1.8 |
| | Ferulic acid | 1.1 ± 0.1 | 23.0 ± 0.9 |
| | Isoferulic acid | ND | 27.5 ± 1.1 |
| Anthocyanins | Cyanidin-3-O- | 5.3 ± 0.3 | 1800 ± 72 |
| | galactoside | | |
| Flavan-3-ols | (+)-Catechin | 11.5 ± 0.9 | 359 ± 14.3 |
| | (−)-Epicatechin | 71.0 ± 4.2 | 4627 ± 185 |
| | Epigalocatechin | ND | 5.5 ± 0.2 |
| Total Phenolics | | 399.1 | 42025.5 |
| detected by | | | |
| LC-MS/MS | | | |

¹Mean ± standard deviation of three replicates; ND, not detected

TABLE 5
The total antioxidant capacity of the two apple skin extracts prepared
from ‘Northern Spy’ apples.

| | Apple | |
|---|---|---|
| Antioxidant Capacity Assay | Skin Extract 1 | Apple Skin Extract 2 |
| Folin-Ciocalteu assay | 6.7 ± 0.6 | 1990 ± 74 |
| (mg gallic acid equivalents | | |
| per L of extract) | | |
| FRAP | 728 ± 32 | 230773 ± 6168 |
| (mg Trolox equivalents per | | |
| L of extract) | | |
| ORAC | 147 ± 0.7 | 19702 ± 150 |
| (mg Trolox equivalents per | | |
| L of extract) | | |
| DPPH (IC₅₀ value) | 129 (r² = 0.9313) | 36.3 (r² = 0.9864) |
| IC₅₀ for Trolox = 178 | | |
| (r² = 0.9802) | | |

¹Mean ± standard deviation of three replicates

TABLE 6
Percent inhibition of heat-induced oxidation of ML, EPA and DHA in
aqueous emulsions by apple skin extracts with comparison to
α-tocopherol, BHT and TBHQ (The oxidation was determined
based on the TBARS formation)

| | Concentration | | | |
|---|---|---|---|---|
| | (μg total | | | |
| | phenolics per | % Inhibition of oxidation | | |
| Antioxidants | mL emulsion) | ML | EPA | DHA |
| Apple Skin | 2000 | 72.6 ± 7.5 | 65.0 ± 4.3 | 59.2 ± 5.0 |
| Extract 1 | 400 | 87.1 ± 7.5 | 72.5 ± 20.3 | 71.4 ± 8.4 |
| | 200 | 93.8 ± 6.9 | 33.0 ± 14.9 | 72.8 ± 7.0 |
| | 40 | 62.9 ± 2.4 | 25.1 ± 8.9 | 26.2 ± 15.3 |
| | 8 | 38.4 ± 1.9 | 15.5 ± 12.5 | 16.6 ± 15.0 |
| Apple Skin | 40 | 83.2 ± 26.2 | 80.6 ± 8.4 | 79.5 ± 13.9 |
| Extract 2 | 20 | 92.5 ± 9.1 | 73.1 ± 17.7 | 86.9 ± 12.1 |
| | 4 | 99.1 ± 11.2 | 89.4 ± 7.5 | 79.0 ± 9.3 |
| | 2 | 78.3 ± 7.1 | 31.2 ± 10.8 | 38.2 ± 8.6 |
| | 0.4 | 23.1 ± 8.6 | 2.4 ± 12.8 | 13.1 ± 18.9 |
| α- | 400 | 1.9 ± 2.2 | 0 | 0 |
| Tocopherol | | | | |
| BHT | 20 | 86.6 ± 1.2 | 59.7 ± 2.7 | 21.7 ± 25.4 |
| TBHQ | 20 | 93.4 ± 11.2 | 71.7 ± 3.6 | 54.2 ± 6.3 |

BHT, butylated hydroxytoluene;
TBHQ, tert-butyl hydroquinone;
Values are mean of two independent experiments of triplicate ± SD.

TABLE 7
Percent inhibition of peroxyl radical-induced oxidation of ML, EPA and
DHA in aqueous emulsion by apple skin extracts with comparison to α-
tocopherol, BHT and TBHQ (The oxidation was determined based on the
TBARS)

| | Concentration | | | |
|---|---|---|---|---|
| | (μg total | | | |
| | phenolics per | % Inhibition of oxidation | | |
| Antioxidants | mL emulsion) | ML | EPA | DHA |
| Apple Skin | 2000 | 79.6 ± 6.2 | 75.79 ± 7.5 | 83.5 ± 11.3 |
| Extract 1 | 400 | 90.5 ± 9.4 | 67.83 ± 13.7 | 67.9 ± 11.6 |
| | 200 | 90.3 ± 9.3 | 61.85 ± 14.3 | 57.7 ± 10.9 |
| | 40 | 23.6 ± 3.9 | 29.34 ± 5.6 | 32.4 ± 10.3 |
| | 8 | 0 | 18.39 ± 10.7 | 30.9 ± 18.8 |
| Apple Skin | 40 | 91.2 ± 5.6 | 92.6 ± 20.4 | 79.4 ± 11.6 |
| Extract 2 | 20 | 89.5 ± 6.3 | 79.9 ± 20.7 | 67.9 ± 10.4 |
| | 4 | 85.3 ± 8.3 | 45.6 ± 19.6 | 45.8 ± 10.7 |
| | 2 | 68.3 ± 4.8 | 22.5 ± 32.5 | 34.4 ± 7.2 |
| | 0.4 | 21.5 ± 12.5 | 4.5 ± 15.5 | 16.1 ± 7.7 |
| α- | 400 | 53.9 ± 7.8 | 0 | 0 |
| Tocopherol | | | | |
| BHT | 20 | 98.6 ± 9.2 | 82.1 ± 9.0 | 65.7 ± 1.8 |
| TBHQ | 20 | 99.1 + 10.2 | 69.2 ± 11.3 | 74.8 ± 13.5 |

BHT, butylated hydroxytoluene;
TBHQ, tert-butyl hydroquinone;
Values are mean of two independent experiments of triplicate ± SD.

TABLE 8
Percent inhibition of UV-induced oxidation of ML, EPA and DHA in
aqueous emulsion by apple skin extracts with comparison to α-tocopherol, BHT
and TBHQ (The oxidation was determined based on the TBARS)

| | Concentration | | | |
|---|---|---|---|---|
| | (μg total | | | |
| | phenolics per | % Inhibition of oxidation | | |
| Antioxidants | mL emulsion) | ML | EPA | DHA |
| Apple Skin Extract 1 | 2000 | 40.4 ± 18.5 | 42.2 ± 16.8 | 38.7 ± 15.3 |
| | 400 | 55.4 ± 17.8 | 70.7 ± 15.1 | 31.1 ± 8.9 |
| | 200 | 54.6 ± 15.5 | 35.3 ± 13.6 | 18.9 ± 7.3 |
| | 40 | 32.6 ± 7.6 | 51.8 ± 13.7 | 12.6 ± 4.0 |
| | 8 | 11.8 ± 11.8 | 33.1 ± 8.1 | 15.6 ± 8.9 |
| Apple Skin Extract 2 | 40 | 49.8 ± 8.1 | 51.5 ± 5.7 | 26.3 ± 13.0 |
| | 20 | 62.9 ± 7.6 | 51.0 ± 15.9 | 25.8 ± 7.6 |
| | 4 | 57.4 ± 10.9 | 27.9 ± 9.2 | 10.7 ± 15.4 |
| | 2 | 37.9 ± 8.2 | 19.4 ± 4.2 | 7.2 ± 0.8 |
| | 0.4 | 20.6 ± 8.2 | 8.2 ± 0.8 | 6.2 ± 0.8 |
| α- | 400 | 22.0 ± 23.3 | 0 | 0 |
| Tocopherol | | | | |
| BHT | 20 | 99.9 ± 26.0 | 69.58 ± 11.1 | 18.49 ± 24.6 |
| TBHQ | 20 | 100 ± 10.4 | 60.50 ± 20.9 | 76.12 ± 4.3 |

BHT, butylated hydroxytoluene;
TBHQ, tert-butyl hydroquinone;
Values are mean of two independent experiments of triplicate ± SD.

TABLE 9
Percent inhibition of heat- and UV-induced oxidation of DHA in
aqueous emulsion by apple skin extracts with comparison to
α-tocopherol, BHT and TBHQ (The oxidation was determined
based on the FTC)

| | Concentration | | |
|---|---|---|---|
| | (μg of total | % Inhibition | |
| | phenolics per | of oxidation | |
| Antioxidants | mL emulsion) | Heat | UV |
| Apple Skin Extract 1 | 2000 | 63.1 ± 0.1 | 57.0 ± 1.3 |
| | 400 | 55.5 ± 0.1 | 49.3 ± 0.2 |
| | 200 | 52.7 ± 0.9 | 40.9 ± 4.5 |
| Apple Skin Extract 2 | 40 | 68.6 ± 0.4 | 73.3 ± 73.3 |
| | 20 | 67.1 ± 0.2 | 65.8 ± 1.7 |
| | 4 | 61.2 ± 1.8 | 64.0 ± 1.7 |
| α-Tocopherol | 400 | 18.3 ± 0.8 | 56.1 ± 1.4 |
| BHT | 20 | 81.9 ± 0.3 | 70.2 ± 1.5 |
| TBHQ | 20 | 74.6 ± 0.1 | 81.7 ± 2.0 |

BHT, butylated hydroxytoluene;
TBHQ, tert-butyl hydroquinone;
Values are mean of two independent experiments of triplicate ± SD.

TABLE 10
Percent inhibition of heat-, peroxyl radical-, and UV-induced oxidation
of bulk fish oil by apple skin extracts with comparison to α-tocopherol
and BHT (The oxidation was determined based on the TBARS)

| | Concentration | | | |
|---|---|---|---|---|
| | (μg of total | | | |
| | phenolics per | % Inhibition of oxidation | | |
| | mL bulk fish | | Peroxyl | |
| Antioxidants | oil) | Heat | radical | UV |
| Apple Skin | 20000 | 58.4 ± 16.1 | 54.6 ± 27.7 | 74.9 ± 5.2 |
| Extract 1 | 4000 | 40.0 ± 4.8 | 28.1 ± 42.0 | 41.5 ± 7.7 |
| | 2000 | 8.3 ± 2.4 | 14.3 ± 11.4 | 17.1 ± 3.1 |
| | 400 | 0 | 9.8 ± 0.3 | 19.9 ± 5.1 |
| Apple Skin | 400 | 93.2 ± 4.2 | 92.1 ± 9.3 | 98.8 ± 3.1 |
| Extract 2 | 200 | 73.6 ± 9.0 | 86.1 ± 0.4 | 97.4 ± 2.0 |
| | 40 | 51.5 ± 12.3 | 69.2 ± 17.5 | 93.2 ± 3.0 |
| | 20 | 0 | 14.9 ± 25.8 | 41.1 ± 36.3 |
| α- | 40000 | 88.6 ± 7.0 | 97.1 ± 5.8 | 92.3 ± 0.1 |
| Tocopherol | 4000 | 78.4 ± 5.8 | 87.2 ± 7.3 | 84.2 ± 0.4 |
| | 400 | 87.0 ± 2.6 | 76.2 ± 16.5 | 83.6 ± 2.1 |
| BHT | 20000 | 99.7 ± 0.0 | 99.7 ± 8.2 | 95.0 ± 1.5 |
| | 2000 | 93.2 ± 3.4 | 97.5 ± 7.9 | 94.1 ± 1.4 |
| | 200 | 46.6 ± 41.7 | 88.7 ± 3.5 | 89.1 ± 0.2 |

BHT, butylated hydroxytoluene;
TBHQ, tert-butyl hydroquinone;
Values are mean of two independent experiments of triplicate ± SD.

TABLE 11
Percent inhibition of heat- and UV-induced oxidation of bulk fish oil
by apple skin extracts with comparison to α-tocopherol, BHT and
TBHQ (The oxidation was determined based on the FTC)

| | |
|---|---|
| Concentration | |
| (μg of total | |
| phenolics per | % Inhibition |
| mL emulsion | of oxidation |

| Antioxidants | or ppm) | Heat | UV |
|---|---|---|---|
| Apple Skin Extract 1 | 20000 | 60.2 ± 0.9 | 44.1 ± 0.1 |
| | 4000 | 42.5 ± 0.8 | 35.5 ± 0.3 |
| | 2000 | 25.3 ± 2.5 | 34.7 ± 0.2 |
| Apple Skin Extract 2 | 400 | 51.0 ± 0.1 | 60.9 ± 0.1 |
| | 200 | 43.8 ± 0.2 | 55.7 ± 0.5 |
| | 40 | 35.6 ± 1.5 | 42.2 ± 1.8 |
| α-Tocopherol | 400 | 89.6 ± 1.1 | 91.0 ± 0.4 |
| BHT | 20 | 86.8 ± 0.1 | 62.5 ± 0.1 |
| TBHQ | 20 | 84.2 ± 0.1 | 79.1 ± 0.1 |

BHT, butylated hydroxytoluene;
TBHQ, tert-butyl hydroquinone;
nd, not determined.
Values are mean of two independent experiments of triplicate ± SD.

TABLE 12
Induction time at 100° C. measured by Rancimat for bulk fish oil
incorporated with different concentrations of apple skin extract 1
and 2 and food antioxidants, α-tocopherol and BHT.

| | Concentration | | |
|---|---|---|---|
| | (μg total | Induction | |
| | phenolics per mL | Time | |
| Antioxidants | fish oil or ppm) | (h) | Stability factor |
| Control | | 0.30 ± 0.01 | |
| Apple Skin Extract 1 | 20000 | 0.75 ± 0.08 | 2.50 |
| | 4000 | 0.59 ± 0.05 | 1.96 |
| | 2000 | 0.48 ± 0.05 | 1.60 |
| | 400 | 0.35 ± 0.02 | 1.22 |
| | 80 | 0.34 ± 0.05 | 1.13 |
| Apple Skin Extract 2 | 800 | 0.44 ± 0.07 | 1.47 |
| | 400 | 0.49 ± 0.02 | 1.63 |
| | 200 | 0.42 ± 0.04 | 1.40 |
| | 40 | 0.36 ± 0.06 | 1.20 |
| | 8 | 0.43 ± 0.04 | 1.43 |
| α-Tocopherol | 400 | 0.46 ± 0.01 | 1.53 |
| BHT | 20 | 0.44 ± 0.03 | 1.46 |

TABLE 13
Induction time at different temperatures measured by Rancimat for
bulk fish oil incorporated with different concentrations of apple skin extract 1 and
2 and food antioxidants, α-tocopherol and BHT.

| | Concentration | | | | | |
|---|---|---|---|---|---|---|
| | (μg of total | | | | | |
| | phenolics per | | | | | |
| | mL fish oil or | Induction Time (h) | | | | |
| Antioxidants | ppm) | 70° C. | 80° C. | 90° C. | 100° C. | 110° C. |
| Control | | 0.49 ± 0.08 | 0.61 ± 0.02 | 0.41 ± 0.01 | 0.30 ± 0.01 | 0.16 ± 0.01 |
| Apple Skin | 400 | 1.59 ± 0.14 | 1.11 ± 0.03 | 0.71 ± 0.05 | 0.59 ± 0.05 | 0.33 ± 0.01 |
| Extract 1 | | | | | | |
| Apple Skin | 400 | 4.34 ± 0.03 | 2.28 ± 0.18 | 0.58 ± 0.03 | 0.49 ± 0.02 | 0.29 ± 0.03 |
| Extract 2 | | | | | | |
| α- | 400 | 4.76 ± 0.17 | 2.40 ± 0.21 | 0.49 ± 0.03 | 0.46 ± 0.01 | 0.26 ± 0.01 |
| Tocopherol | | | | | | |
| BHT | 20 | 1.34 ± 0.05 | 1.00 ± 0.03 | 0.49 ± 0.01 | 0.44 ± 0.03 | 0.15 ± 0.01 |

TABLE 14
Estimated Induction time at room temperature (25° C.)

| | Concentration (μg of | | |
|---|---|---|---|
| | total phenolics per | Induction Time | |
| Antioxidants | mL fish oil or ppm) | (h) | R² |
| Control | — | 18.98 | 0.6532 |
| Apple Skin Extract 1 | 400 | 23.15 | 0.6574 |
| Apple Skin Extract 2 | 400 | 64.27 | 0.6882 |
| α-Tocopherol | 400 | 69.89 | 0.6921 |
| BHT | 20 | 69.79 | 0.6174 |

### FULL CITATIONS FOR REFERENCES REFERRED TO IN THE SPECIFICATION

Amarowicz, R., Naczk, M. Shahidi, F. (2000) Antioxidant activity of various fractions of non-tannin phenolics of canola hulls. J. Agri. Food chem., 48, 2755-2759.
Benzie I, Strain J. 1996. The ferric reducing ability of plasma (FRAP) as a measure of “antioxidant power”: the FRAP assay. Anal Biochem 239(1):70-76.
Boadi W Y, Iyere P A, Adunyah S E. 2003. Effect of quercetin and genistein on copper- and iron-induced lipid peroxidation in methyl linolenate. J Appl Toxicol 23(5):363-369.
Bottenweck, A. A. M., Verhagen, H., Goldbohm, R. A., Kleinjans, J., Brandt, P. A. V. D. (2000) Intake of butylated hydroxyanisole and butylated hydroxytoluene and stomach cancer risk: results from analyses in the Netherlands cohort study, Food Chemistry and Toxicology 38, 599-605.
Cao G, Alessio H, Cutler R. 1993. Oxygen-radical absorbance capacity assay for antioxidants. Free Radic Biol Med 14(3):303-311.
Drogoudi P D, Michailidis Z, Pantelidis G. 2008. Peel and flesh antioxidant content and harvest quality characteristics of seven apple cultivars. Sci Horticult 115(2):149-53.
Eberhardt, M. V., Lee, C. Y., Liu, R. H., (2000) Antioxidant activity of fresh apples. Nature 405, 903-904.
Erkan, N., Ayranci, G., Ayranci, E. (2008) Antioxidant activity of rosemary (Rosmarinus Officinalis L.) extract, blackseed (Nigella sativa L.) essential oil, carnosic acid, rosmarinic acid and sesamol. Food chemistry 110, 76-82.
Esterbauer, H.; Schaur, R. J.; Zollner, H. (1990) Chemistry and biochemistry of 4-hydroxynonenal, malonaldehyde and related aldehydes. Free Radical Biology and Medicine. 11, 81-128.
Fang, J.; Vaca, L.; Valsta, L.; Mutanen, M. (1996) Determination of DNA adducts of malonaldehyde in humans: Effects of dietary fatty acid composition. Carcinogenesis. 17, 1035-1040.
Friedrich W, Eberhardt A, Galensa R. 2000. Investigation of proanthocyanidins by HPLC with electrospray ionization mass spectrometry. European food research and technology=Zeitschrift für Lebensmittel-Untersuchung und-Forschung A 211(1):56-64.
Frankel, E. N., Huang, S.-W., Aeschbach, R. and Prior, E. (1994) Antioxidant activity of rosemary extract and its constituents, Carnosic acid, Carnosol and Rosemarinic acid in bulk oil and oil-in-water emulsion. J Agri Food Chem 44:131-135.
Hodges D M, DeLong J M, Formey C F, Prange R K. 1999. Improving the thiobarbituric acid-reactive-substances assay for estimating lipid peroxidation in plant tissues containing anthocyanin and other interfering compounds. Planta 207(4):604-11.
Huang D, Ou B, Hampsch-Woodill M, Flanagan J A, Prior R L. 2002. High-throughput assay of oxygen radical absorbance capacity (ORAC) using a multichannel liquid handling system coupled with a microplate fluorescence reader in 96-well format. J Agric Food Chem 50(16):4437-44.
Iqbal S, Bhanger M I. 2007. Stabilization of sunflower oil by garlic extract during accelerated storage. Food Chem 100(1):246-54.
Katsandis E, Addis P B, Epley R J, Futcher R G. 1997. Evaluation of the antioxidant properties of barley flour and wild rice in uncooked and precooked ground beef patties. J Food Service Systems, 10(1):9-22.
Kaur, C., Kapoor, H. C. (2001) Antioxidants in fruits and vegetables-millennium's health. International J Food Sci Tec 36, 703-725.
Kondo S, Tsuda K, Muto N, Ueda J. 2002. Antioxidative activity of apple skin or flesh extracts associated with fruit development on selected apple cultivars. Sci Horticult 96(1-4):177.
Kremer, J. M. (2000) n-3 Fatty acid supplements in rheumatoid arthritis, American Journal of Clinical Nutrition 71, 349S-351S.
Lemanska K, Szymusiak H, Tyrakowska B, Zielinski R, Soffers A E M F, Rietjens I M C M. 2001. The influence of pH on antioxidant properties and the mechanism of antioxidant action of hydroxyflavones. Free Radic Biol Med 31(7): 869-881.
Montero P, Giménez B, Pérez-Mateos M, Gómez-Guillén MC. 2005 Oxidation stability of muscle with quercetin and rosemary during thermal and high-pressure gelation. Food Chem 93(1):17-23.
Nawar, W. 1996. Lipids. In: Food chemistry 3^{rd} edition. Fennema, O. (ed.) Marcel Dekker, Inc.
Okuda S, McClements D J, Decker E A. 2005. Impact of lipid physical state on the oxidation of methyl linolenate in oil-in-water emulsions. J Agric Food Chem 53(24):9624-8.
Osawa T, Namiki M. 1981. Novel type of antioxidant isolated from leaf wax of Eucalyptus leaves. Agric Biol Chem 45: 735-739.
Ozsoy, N., Can, A., Yanardag, R., Akev, N (2008) Antioxidant activity of Smilax excelsa L. leaf extracts. Food Chemistry, 110: 571-583.
Pazos, M., Gallardo, J. M., Torres, J. L., Medina, I. (2005) Activity of grape polyphenols as inhibitors of the oxidation of fish lipids and frozen fish muscle. Food Chem 92, 547-557.
Prior R L, Wu X, Schaich K. 2005. Standardized methods for the determination of antioxidant capacity and phenolics in foods and dietary supplements. J Agric Food Chem 53(10):4290-302.
Rupasinghe H P V. 2003. Using change for success: Fruit-based bio-product research at the Nova Scotia Agricultural College. Annual Report 2003 of the Nova Scotia Fruit Growers' Association, Kentville, NS, Canada. p. 66-69.
Rupasinghe, H. P. V.; Wang, L.; Huber, G. M.; Pitts, N. L. (2008) Effect of baking on dietary fibre and phenolics of muffins incorporated with apple skin powder. Food Chemistry. 2008, 107, 1217-1224.
Simopoulos, A. (2002) The importance of the ratio of omega-6/omega-3 essential fatty acids. Biomedicine & Pharmacotherapy. 2002, 56, 365-379.
Singleton V L, Orthofer R, Lamuela-Raventos R M. 1999. Analysis of total phenols and other oxidation substrates and antioxidants by means of Folin-Ciocalteu reagent. Methods Enzymol 299: 152-178.
Tabata, H., Katsube, T. Tsuma, T. Ohta, Y., Imawaka, N., Utsumi, T. (2008) Isolation and evaluation of the radical-scavenging activity of the antioxidants in the leaves of an edible plant, Mallotus Japonicus. Food Chemistry 109, 64-71.
Tsimidou, M., Papavergou, E., Boskon, D. (1995) Evaluation of oregano antioxidant activity in mackerel oil. Food Research International 28, 431-433.
Vidal S, Leigh F, Guyot S, Marnet N, Kwiatkowski M, Gawel R, Cheyneir V, Waters E. 2003. The mouth-feel properties of grape and apple proanthocyanidins in a wine-like medium. J Sci Food Agric 83(6):564-573.
Vinson, J. A., Su, X., Zubik, L., Bose. (2001) Phenolic antioxidant quantity and quality in foods: fruits. J Agric food Chem 49:5315-5321.
Wanasundara U N, Shahidi F. 1998. Antioxidant and pro-oxidant activity of green tea extracts in marine oils. Food Chem 63(3):335-42.
Wang, C. C., Harris, W. S., Chung, M., Lichtenstein, A. H., Balk, E. M., Kupelnick, B. et al., (2006) n-3 Fatty acids from fish or fish-oil supplements, but not alpha-linolenic acid, benefit cardiovascular disease outcomes in primary- and secondary-prevention studies: a systematic review, American Journal of Clinical Nutrition 84, 5-17.
Wettasinghe M, Shahidi F. 1999. Antioxidant and free radical-scavenging properties of ethanolic extracts of defatted borage (Borago officinalis L.) seeds. Food Chem 67(4): 399-414.
Wigmore, S. J., Ross, J. A., Falconer, J. S., Plester, C. E., Tisdale, M. J., Carter, D. C., (1996) The effect of polyunsaturated fatty acids on the progress of cachexia in patients with pancreatic cancer, Nutrition 12 S27-S30.
Wolfe K L, Liu R H. 2003. Apple peels as a value-added food ingredient. J Agric Food Chem 51(6):1676-83.
Wolfe, K., Wu, X., Liu, R. H., (2003) Antioxidant activity of apple peels. Journal of Agricultural and Food Chemistry 51, 609-614.

1. A method of preventing or inhibiting the oxidation of polyunsaturated fatty acids and/or lipids comprising contacting the polyunsaturated fatty acids and/or lipids with an effective amount of an extract comprising phenolic compounds from apple skins.

2. The method according to claim 1, wherein the effective amount of the extract comprising phenolic compounds from apple skins is an amount to provide about 1 ppm to about 20,000 ppm of total phenolics in a product comprising the polyunsaturated fatty acids (PUFA) and/or lipids.

3. The method according to claim 1 wherein the extract comprising phenolic compounds has been treated under conditions to remove sugar compounds.

4. The method according to claim 1, wherein the extract comprising phenolic compounds has been treated under conditions to remove lipids, carontenoids, chlorophylls and/or proanthocyanidins.

5. The method according to claim 1, wherein the polyunsaturated fatty acids and/or lipids are comprised in any nutraceutical (natural health product) or cosmetic product containing polyunsaturated fatty acids and/or lipids.

6. (canceled)

7. (canceled)

8. The method according to claim 1, wherein the phenolic compounds are extracted from the apple skins using a food-grade solvent.

9. (canceled)

10. The method according to claim 1, wherein the extract further comprises food-grade carrier.

11. (canceled)

12. A process for extracting apple phenolic compounds from apples, comprising:
(a) obtaining a sample of apple peels,
(b) optionally dehydrating the peels and converting the peels into a powder;
(c) extracting the peels with a food-grade solvent under conditions to extract the apple phenolic compounds into the solvent; and
(d) removing solids from the extract of (c) to provide a stock solution of apple phenolic compounds.

13. (canceled)

14. (canceled)

15. The process according to claim 12, wherein the conditions to extract the apple phenolic compounds into the solvent comprise sonicating for about 5 minutes to 2 hours.

16. (canceled)

17. The process according to claim 12, wherein the stock solution of extracted apple phenolic compounds is reduced to dryness to provide a solid concentrate of apple phenolic compounds.

18. The process according to claim 17, wherein the solid concentrate is taken up into water to provide an aqueous solution having insoluble suspended material and this insoluble material is removed to provide a clear aqueous solution of extracted apple phenolic compounds.

19. The process according to claim 12, further comprising removing proanthocyanidins, lipids, carontenoids and/or chlorophylls from the peels by extracting the peels with hexane and/or by extracting the peels with a mixture of acetone, water and acetic acid.

20. The process according to claim 19, wherein the stock solution of apple phenolic compounds is reduced to dryness to provide a solid concentrate of extracted apple phenolic compounds.

21. The process according to claim 20, wherein the solid concentrate is further taken up into water to provide an aqueous solution having insoluble suspended material and this insoluble material is removed to provide a clear aqueous solution of extracted apple phenolic compounds.

22. The process according to claim 21, wherein the stock solution of extracted apple phenolic compounds, the solid concentrate of extracted apple phenolic compounds or the aqueous solution of extracted apple phenolic compounds is treated to remove sugar compounds.

23. The process according to claim 22, wherein a food-grade carrier is added to the stock solution of extracted apple phenolic compounds, the solid concentrate of extracted apple phenolic compounds or the aqueous solution of extracted apple phenolic compounds or the sugar removed versions thereof.

24. (canceled)

25. A animal or human food product comprising an extract of phenolic compounds from apples, wherein the extract is prepared using the process according of claim 12.

26. The food product according to claim 25, wherein the product is in the form of emulsions, oils, liquids, solids, creams, or a microencapsulated product.

27. The food product according to claim 26 in the form of an oil in water emulsion or a bulk oil.

28. The food product according to claim 27, wherein the bulk oil is fish oil.
